(19) United States
(12) Patent Application Publication
(10) Pub. No.: US 2025/0312453 A1
(43) Pub. Date: Oct. 9, 2025

(54) ANTIBODY TARGETING CCR8 AND ITS APPLICATIONS

(71) Applicant: BRISTAR IMMUNOTECH LIMITED, Beijing (CN)

(72) Inventors: Yang LI, Beijing (CN); Dongmei YANG, Beijing (CN); Zhe WAN, Beijing (CN)

(21) Appl. No.: 19/088,850

(22) Filed: Mar. 24, 2025

(30) Foreign Application Priority Data
Apr. 9, 2024 (CN) 202410424073.7

(51) Int. Cl. A61K 40/42; A61K 40/11; A61K 40/31; A61P 35/00; C07K 14/54; C07K 16/28; C07K 16/30

(52) U.S. Cl. CPC A61K 40/4219; A61K 40/11; A61K 40/31; A61K 40/4202; A61K 40/4234; A61K 40/4255; A61P 35/00; C07K 14/5443; C07K 16/28; C07K 16/2866; C07K 16/30; C07K 2317/569; C07K 2317/92

(57) ABSTRACT

The present invention belongs to the field of biomedicine. Specifically, it provides an antibody targeting CCR8 and its applications. More particularly, the present invention offers an antibody targeting CCR8, STAR and CAR derived from the antibody, therapeutic immune cells containing the STAR or CAR, and their applications in disease treatment.

### CROSS-REFERENCE TO RELATED APPLICATIONS

The present application claims the priority benefit of Chinese Patent Application No. 202410424073.7, filed Apr. 9, 2024, which is herein incorporated by reference in its entirety.

### REFERENCE TO AN ELECTRONIC SEQUENCE LISTING

The contents of the electronic sequence listing (8575-0082_Sequence listing.xml; Size: 69351 bytes; and Date of Creation: Mar. 11, 2025) is herein incorporated by reference in its entirety.

### TECHNICAL FIELD

The present invention relates to the field of biomedicine, particularly to the field of cell therapy. Specifically, it relates to a Synthetic T-Cell Receptor and Antigen Receptor (STAR) that targets receptors on immunosuppressive immune cells and tumor antigens. The present invention also relates to vectors containing the receptors, T cells containing the receptors, and methods for their preparation.

### BACKGROUND

Chimeric antigen receptor T-cell (CAR-T) therapy is an anti-cancer immunotherapy that has achieved favorable therapeutic effects in recent years. Unlike the way natural T-cell recognize tumor cells, the CAR-T cells do not depend on MHC molecules (such as HLA) for tumor cells recognition. The CAR molecule mainly consists of an extracellular region responsible for recognizing the target antigen, a transmembrane region, and an intracellular region responsible for transmitting T-cell activation signals after receiving stimulation. When the CAR molecule is transfected into the patient's T cells through transfection technology, the patient's T cells express the tumor antigen receptor. After purification and expansion, CAR-T cells have been used to treat B-cell lymphoma and hematological tumors. However, CAR-T therapy encounters difficulties when used to treat solid tumors. There are multiple reasons for the poor efficacy of CAR-T cell therapy in solid tumor treatment. One important reason is that the function of CAR-T cells is inhibited in the tumor microenvironment, and T cells are prone to exhaustion and apoptosis. Recent studies have shown that the inhibitory immune cells (such as MDSC and Treg cells) present in the tumor microenvironment have a strong inhibitory effect on various tumor immunotherapies, including cell therapy, which greatly limits its therapeutic potential.

The T-cell receptor (TCR) complex molecule contains multiple chains. The TCR α chain and TCR β chain are responsible for recognizing MHC-peptide molecules, and the other six CD3 subunits bind to the TCR α/β chains and function in signal transduction. The natural TCR complex contains 10 ITAM signal sequences, and theoretically can transmit stronger signals than CAR. Previous studies have shown that although the signal of TCR is transmitted more slowly than that of CAR, the TCR signal is more persistent. Therefore, utilizing the signal-transduction function of the natural TCR can alleviate T-cell dysfunction and enable it to better exert its anti-solid tumor effect. However, the recognition of tumor cells by TCR depends on MHC molecules, and its affinity is also lower than that of CAR.

The ectodomain of TCR is very similar to the Fab domain of an antibody, so the variable region sequence of TCR can be replaced by a variable region sequence of an antibody (e.g., scFv), so as to obtain a Synthetic T-Cell Receptor and Antigen Receptor (STAR). STAR combines the advantages of TCR and CAR simultaneously, which not only has antibody specificity, but also has superior signal transduction function of a natural TCR on mediating T-cell activation, and can mediate complete T-cell activation. It has significant improvements in safety and efficacy, thus becoming a promising new type of cell immunotherapy.

However, STAR derived from the natural TCR still cannot overcome the drawbacks of CAR-T cells, such as the inhibition of function in the tumor microenvironment and the tendency of T cells to exhaustion and apoptosis. Regulatory T (Treg) cells expressing Foxp3 inhibit abnormal immune responses to self-antigens and are also a key cellular component in the tumor microenvironment that suppresses anti-tumor immune responses. The infiltration of a large number of Treg cells into tumor tissues is often associated with poor prognosis. Increasing evidence shows that the depletion of Treg cells can enhance anti-tumor immune responses. On the other hand, the systemic depletion of Treg cells may also cause harmful autoimmune reactions in the body (such as anti-CTLA-4 mAb, IL2-Fc, and anti-CCR4 mAb). Therefore, a more ideal tumor immunotherapy strategy involves specifically targeting tumor-infiltrating Treg cells with immunosuppressive activity, while without targeting inflammatory Treg cells and non-Treg type T cells.

CCR8 is a member of the chemokine receptor subfamily and is a seven-transmembrane G-protein-coupled receptor. CCR8 is specifically expressed on tumor-infiltrating regulatory T cells (Treg), but is minimally expressed on peripheral blood Treg or normal tissues. It is the receptor for the chemokine CCLI and is involved in the recruitment of Tregs and Th2 cells to inflammatory and tumor sites. CCR8 is commonly upregulated in tumor tissues and exhibits a high correlation with Foxp3. The expression of CCR8 and Foxp3 is associated with survival rates and disease stages in various cancers. Antibodies targeting CCR8 can specifically eliminate tumor-infiltrating, immunosuppressive Treg cells and inhibit tumor growth, without targeting inflammatory Treg cells, peripheral Treg cells, or effector T cells (Teffs) in tumor tissues.

Building on the advantages of STAR and the specific expression of CCR8 on the surface of tumor-infiltrating Treg cells. In the present invention, the variable region of one chain of TCR is replaced with a CCR8-targeting antibody or ligand that binds to CCR8 on the surface of Treg cells, and the variable region of the other chain of TCR is replaced with an antibody sequence targeting tumor antigens (e.g., scFv or VHH). Thus, a novel STAR that can target both CCR8 and tumor antigens is designed, constructed and expressed. Since the immunosuppressive Treg cells are eliminated, the targeted killing of tumors by immune cells (such as T cells, NK cells, macrophages, etc.) can be enhanced, thereby further improving the efficacy of cell-based therapies.

### SUMMARY

The present invention relates to a single-domain antibody that specifically binds to CCR8 and its application in Synthetic T-cell Receptor and Antigen Receptor (STAR) and Chimeric Antigen Receptor (CAR). The single-domain antibody contains specific CDR1, CDR2, and CDR3 sequences, enabling it to bind to CCR8 with high efficiency and specificity. Based on this single-domain antibody, the present invention further develops various STAR and CAR structures targeting CCR8, which can be used to treat multiple CCR8-related diseases.

The core content of the present invention includes the following aspects:

Development of Single-Domain Antibodies: The present invention provides a single-domain antibody that specifically binds to CCR8, comprising specific CDR1, CDR2 and CDR3 sequences. For example, the CDR1, CDR2 and CDR3 sequences are selected from CDR1, CDR2 and CDR3 in SEQ ID NO:8, wherein the antibody is a murine-derived antibody or a humanized antibody, with high specificity and affinity.

Synthetic T-cell Receptor and Antigen Receptor (STAR): The present invention provides a STAR targeting CCR8, the antigen-binding region of the STAR contains the single-domain antibody. The STAR structure can include one or more antigen-binding regions and can be combined with antibodies or antigen-binding fragments that specifically bind to other antigens (e.g., MSLN or Claudin18.2) to form dual-targeting structures.

Chimeric Antigen Receptor (CAR): The present invention provides a CAR targeting CCR8, wherein an extracellular antigen-binding region of the CAR contains the single-domain antibody. The CAR structure can also include antigen-binding regions that specifically bind to other antigens to achieve multi-targeting therapy.

Nucleic Acid Molecules and Expression Vectors: The present invention provides nucleic acid molecules encoding the single-domain antibody, STAR, and CAR, as well as expression vectors containing these nucleic acid molecules. These expression vectors can be used to express the corresponding protein structures in host cells.

Host Cells and Therapeutic Immune Cells: The present invention provides cells expressing the single-domain antibody, STAR, or CAR obtained by transforming host cells. These cells can be further used to prepare therapeutic immune cells, such as T cells or NK cells.

Pharmaceutical Compositions and Therapeutic Uses: The present invention provides pharmaceutical compositions containing the single-domain antibody, STAR, CAR, or therapeutic immune cells for treating various CCR8-related diseases, particularly hematologic tumors and solid tumors.

The single-domain antibody, STAR, and CAR structures provided in the present invention exhibit high specificity and affinity, effectively targeting CCR8. The combination with other antigen-binding regions can further enhance therapeutic efficacy. Through the development of nucleic acid molecules and expression vectors, the present invention provides a foundation for large-scale production and application. Additionally, the present invention covers the use of these structures in pharmaceutical compositions, offering new strategies for treating various diseases.

In a first aspect of the present invention, provided is a single-domain antibody specifically binding to CCR8, comprising CDR1, CDR2, and CDR3 sequences selected from CDR1, CDR2 and CDR3 in SEQ ID NO:8.

In some embodiments, the single-domain antibody comprises the CDR1 has an amino acid sequence set forth in SEQ ID NO: 5, wherein the CDR2 has an amino acid sequence set forth in SEQ ID NO: 6, wherein the CDR3 has an amino acid sequence set forth in SEQ ID NO: 7.

In some embodiments, the single-domain antibody comprises the amino acid sequence has at least 80%, preferably at least 90%, more preferably at least 95%, and even more preferably at least 99% identity to SEQ ID NO: 8, preferably, wherein the amino acid sequence shown in SEQ ID NO: 8.

In some embodiments, the single-domain antibody is a murine-derived antibody or a humanized antibody.

In second aspect of the present invention, provided is a synthetic T-cell receptor and antigen receptor (STAR) targeting CCR8, where the STAR comprises the single-domain antibody specifically binding to CCR8.

In some embodiments, the STAR comprises a first peptide chain and a second peptide chain:
i) the first peptide chain comprises a first target-binding region and a first constant region, and the second peptide chain comprises a second target-binding region and a second constant region; or,
ii) the first peptide chain comprises a first constant region, the second peptide chain comprises a second constant region, and the first peptide chain or the second peptide chain comprises a first target-binding region;
wherein the first target-binding region and/or the second target-binding region comprises one or more antigen-binding regions, and the multiple antigen-binding regions are the same or different;
the antigen-binding region in the first target-binding region and/or the second target-binding region comprises the CCR8 single-domain antibody.

In some embodiments, the STAR comprises the antigen-binding region in the first target-binding region and/or the second target-binding region further comprises an antibody or antigen-binding fragment specifically binding to MSLN or Claudin18.2;
preferably, the antibody or antigen-binding fragment is selected from scFv, nanobody, or single-domain antibody.

In some embodiments, the STAR comprises any one of the following groups:
a) the first peptide chain comprises a first constant region; the second peptide chain sequentially comprises, from the N-terminus to the C-terminus, at least one CCR8 single-domain antibody, and a second constant region;
b) the first peptide chain comprises, from the N-terminus to the C-terminus, the CCR8 single-domain antibody, and a first constant region; the second peptide chain sequentially comprises, from the N-terminus to the C-terminus, an antibody or antigen-binding fragment specifically binding to MSLN or Claudin18.2, and a second constant region;
c) the first peptide chain comprises a first constant region; the second peptide chain comprises, from the N-terminus to the C-terminus, at least one CCR8 single-domain antibody, an antibody or an antigen-binding fragment specifically binding to MSLN or Claudin18.2, and a second constant region;

In the each of the above group a)-c), the first constant region of the first peptide chain is a TCRα chain constant region or a TCRβ chain constant region, and the second constant region of the second peptide chain is a TCRβ chain constant region or a TCRα chain constant region; the constant regions of the first peptide chain and the second peptide chain are not simultaneously TCRα chain constant regions, nor are they simultaneously TCRβ chain constant regions.

In some embodiments, the antibody or antigen-binding fragment specifically binding to MSLN comprises CDR1 as shown in SEQ ID NO:31, CDR2 as shown in SEQ ID NO:32, and CDR3 as shown in SEQ ID NO:33; preferably, the antibody or antigen-binding fragment specifically binding to MSLN comprises a single-domain antibody with the amino acid sequence shown in SEQ ID NO:34; and the antibody or antigen-binding fragment specifically binding to Claudin18.2 comprises CDR1 as shown in SEQ ID NO:41, CDR2 as shown in SEQ ID NO:42, and CDR3 as shown in SEQ ID NO:43; preferably, the antibody or antigen-binding fragment specifically binding to Claudin18.2 comprises a single-domain antibody with the amino acid sequence shown in SEQ ID NO:44.

In some embodiments, the first constant region is a TCRα chain constant region or a TCRβ chain constant region, preferably a modified TCRα chain constant region or TCRβ chain constant region; the second constant region is a TCRα chain constant region or a TCRβ chain constant region, preferably a modified TCRα chain constant region or TCRβ chain constant region; preferably, the TCRα chain constant region is selected from the constant region of a wild-type human or wild-type mouse TCRα chain; preferably, the TCRβ chain constant region is selected from the constant region of a wild-type human or wild-type mouse TCRβ chain.

In some embodiments, the modified TCRα chain constant region is derived from a mouse TCRα chain constant region, the amino acids at positions 6, 13, 15-18, 48, 112, 114, 115, 122, 136, and 137 comprise one or more modifications, as compared to the wild-type mouse TCRα chain constant region, wherein the modifications are amino acid substitutions or deletions;

the modified TCRβ chain constant region is derived from a mouse TCRβ chain constant region, the amino acids at positions 3, 6, 9, 11, 12, 17, 21-25, 56, 150, 162-172, 168, or 170 comprise one or more modifications, as compared to the wild-type mouse TCRβ chain constant region, wherein the modifications are amino acid substitutions or deletions; or

In some embodiments, the modified TCRα chain constant region is derived from a human TCRα chain constant region, the amino acids at positions 47, 90-93, 115, and 118 comprise one or more modifications, as compared to the wild-type human TCRα chain constant region, wherein the modifications are amino acid substitutions or deletions; and/or
the modified TCRβ chain constant region is derived from a human TCRβ chain constant region, the amino acids at positions 17, 21, 56, 132, 135, and 138 comprise one or more modifications, as compared to the wild-type human TCRβ chain constant region, wherein the modifications are amino acid substitutions or deletions.

In some embodiments, the modified TCRα chain constant region is derived from a wild-type mouse TCRα chain constant region, as compared to the wild-type mouse TCRα chain constant region, and contains amino acid mutations selected from the following group or combinations thereof:
i) the amino acid threonine (T) at position 48 is mutated to Cysteine (C),
ii) the amino acid serine(S) at position 112 is mutated to leucine (L), the amino acid methionine (M) at position 114 is mutated to isoleucine (I), and/or the amino acid Glycine (G) at position 115 is mutated to valine (V),
iii) the amino acid E at position 6 is substituted by D, and the amino acid K at position 13 is substituted by R, the amino acids at positions 15-18 are deleted;
iv) the amino acid K at position 122 is substituted by R;
v) and lacks the endodomain of the constant region, for example, the amino acids at positions 136-137 are deleted;
and/or, the modified TCRβ chain constant region is derived from a wild-type mouse TCRβ chain constant region, as compared to the wild-type mouse TCRβ chain constant region, and contains amino acid mutations selected from the following group or combinations thereof:
i) the amino acid serine(S) at position 56 is mutated to cysteine (C),
ii) the amino acids at positions 150, 168, and 170 are all substituted by R,
iii) the amino acid R at position 3 is substituted by K, the amino acid T at position 6 is substituted by F, K at position 9 is substituted by E, S at position 11 is substituted by A, and L at position 12 is substituted by V, and/or amino acids at positions 17, 21-25 are deleted,
iv) and lacks the endodomain of the constant region, for example, the amino acids at positions 167-172 are deleted.

In some embodiments, the modified TCRα chain constant region is derived from a wild-type human TCRα chain constant region, as compared to the wild-type human TCRα chain constant region, and contains amino acid mutations selected from the following group or combinations thereof:
i) the amino acid threonine (T) at position 47 is mutated to Cysteine (C),
ii) the amino acid P at position 90 is mutated to S, the amino acid E at position 91 is mutated to D, the amino acid S at position 92 is mutated to V, the amino acid S at position 93 is mutated to P,
iii) the amino acid S at position 115 is mutated to L, the amino acid G at position 118 is mutated to V,
and/or, the modified TCRβ chain constant region is derived from a wild-type human TCRβ chain constant region, as compared to the wild-type human TCRβ chain constant region, and contains amino acid mutations selected from the following group or combinations thereof:
i) the amino acid serine(S) at position 56 is mutated to cysteine (C),
ii) the amino acid E at position 17 is substituted by K, the amino acid S at position 21 is substituted by A, F at position 132 is substituted by I, E at position 135 is substituted by A, and/or Q at position 138 is substituted by H.

In some embodiments, the modified TCRα chain constant region comprises an amino acid sequence shown in one of SEQ ID NOs: 11-15, and/or the modified TCRβ chain constant region comprises an amino acid sequence shown in one of SEQ ID NOs: 18-22.

In some embodiments, the first peptide chain and/or the second peptide chain has at least one exogenous intracellular functional domain linked to its C-terminus to, such as the endodomain of a co-stimulatory molecule, preferably an endodomain of OX40, more preferably, the endodomain of OX40 comprises the amino acid sequence of SEQ ID NO:23.

In some embodiments, wherein the exogenous intracellular functional domain is linked directly or via a linker to the C-terminus of the constant region of the first peptide chain and/or the second peptide chain, preferably, the exogenous intracellular functional domain is linked to the C-terminus of the constant region of the first peptide chain and/or the second peptide chain whose endodomain is deleted, through a linker, preferably, the linker is a (G4S)n linker or an (EAAAK)n linker, where n represents an integer from 1 to 10, preferably, n is 3 or 4.

In some embodiments, the STAR is co-expressed with a membrane-bound IL-15 protein (mbIL-15).

In some embodiments, wherein:
a) the STAR comprises a first peptide chain shown in SEQ ID NO:48 and a second peptide chain shown in SEQ ID NO:47;
b) the STAR comprises a first peptide chain shown in SEQ ID NO:50 and a second peptide chain shown in SEQ ID NO:49;
c) the STAR comprises a first peptide chain shown in SEQ ID NO:52 and a second peptide chain shown in SEQ ID NO:51;
d) the STAR comprises a first peptide chain shown in SEQ ID NO:54 and a second peptide chain shown in SEQ ID NO:53;
e) the STAR comprises a first peptide chain shown in SEQ ID NO:56 and a second peptide chain shown in SEQ ID NO:55; or
f) the STAR comprises a first peptide chain shown in SEQ ID NO:58 and a second peptide chain shown in SEQ ID NO:57.

In some embodiments, wherein:
i) the amino acid sequence of IL-15 is shown in SEQ ID NO: 27;
ii) the amino acid sequence of the extracellular domain of IL-15Rα is shown in SEQ ID NO: 28;
iii) the amino acid sequence of the linker connecting the extracellular domain of IL-15Rα to IL-15 is shown in SEQ ID NO: 24-25, 29; and/or
iv) the amino acid sequence of mbIL-15 is shown in SEQ ID NO: 30.

In third aspect of the present invention, provided is a chimeric antigen receptor (CAR) targeting CCR8, comprising an extracellular antigen-binding region, wherein the extracellular antigen-binding region comprises the CCR8 single-domain antibody, and the CAR sequentially comprises, from the N-terminus to the C-terminus, an extracellular antigen-binding region, a hinge region, a transmembrane domain, a co-stimulatory domain, and an intracellular signal-transduction domain.

In some embodiments, the extracellular antigen-binding region further comprises an antigen-binding region specifically binding to another antigen, preferably, the antigen-binding region specifically binding to the other antigen comprises a single-chain antibody (scFv) or single-domain antibody specifically binding to the other antigen.

In some embodiments, the other antigen is Claudin18.2, preferably, the antigen-binding region specifically binding to Claudin18.2 comprises CDR1 as shown in SEQ ID NO:41, CDR2 as shown in SEQ ID NO:42, and CDR3 as shown in SEQ ID NO:43, preferably, the antigen-binding region specifically binding to Claudin18.2 comprises the amino acid sequence shown in SEQ ID NO: 44;
or, the other antigen is MSLN, preferably, the antigen-binding region specifically binding to MSLN comprises CDR1 as shown in SEQ ID NO:31, CDR2 as shown in SEQ ID NO:32, and CDR3 as shown in SEQ ID NO:33, preferably, the antigen-binding region specifically binding to MSLN comprises the amino acid sequence shown in SEQ ID NO:34.

In some embodiments, the CAR comprises an amino acid sequence shown in any one of SEQ ID NOs: 59-64.

In fourth aspect of the present invention, provided is an isolated nucleic acid molecule encoding the aforementioned single-domain antibody, or the aforementioned STAR, or the aforementioned CAR.

In fifth aspect of the present invention, provided is an expression vector comprising the aforementioned nucleic acid molecule, wherein the nucleic acid molecule is operably linked to an expression regulatory element such as a promoter.

In some embodiments, wherein the expression vector comprises:
1) a coding nucleotide sequence of a fusion polypeptide of the first peptide chain of the STAR and the second peptide chain of the STAR connected by a self-cleaving peptide; or
2) a coding nucleotide sequence of a fusion polypeptide of the first peptide chain of the STAR, the second peptide chain of the STAR, and mbIL-15 connected by a self-cleaving peptide.

In some embodiments, the self-cleaving peptide is a 2A peptide, preferably, the self-cleaving peptide is a Furin-2A peptide, such as the Furin-P2A peptide shown in SEQ ID NO:26.

In sixth aspect of the present invention, provided is a host cell obtained by transforming a cell with the aforementioned nucleic acid molecule or the aforementioned expression vector.

In seventh aspect of the present invention, provided is an isolated therapeutic immune cell comprising the aforementioned STAR or the aforementioned CAR.

In some embodiments, the immune cell is a T cell or natural killer (NK) cell, preferably a T cell.

In eighth aspect of the present invention, provided is a method for preparing the aforementioned therapeutic immune cell, comprising:
Step 1) providing a starting immune cell;
Step 2) introducing the aforementioned expression vector into the starting immune cell; and
Step 3) harvesting the immune cell obtained in Step 2.

In ninth aspect of the present invention, provided is a method for producing a single-domain antibody specifically binding to CCR8, comprising:
(i) culturing the aforementioned host cell under conditions suitable for expression of the nucleic acid molecule or expression vector, and
(ii) isolating and purifying the single-domain antibody specifically binding to CCR8 expressed by the host cell.

In tenth aspect of the present invention, provided is a pharmaceutical composition comprising the aforementioned single-domain antibody, the aforementioned STAR, the aforementioned CAR, the aforementioned therapeutic immune cell, and/or the aforementioned expression vector, and a pharmaceutically acceptable carrier, preferably for treating a disease in a subject.

In eleventh aspect of the present invention, provided is the use of the aforementioned single-domain antibody, the aforementioned STAR, the aforementioned CAR, the aforementioned therapeutic immune cell, and/or the aforementioned expression vector and/or the aforementioned pharmaceutical composition in the preparation of a medicament for treating a disease in a subject.

In some embodiments, the disease is a CCR8-related disease, such as a CCR8-related autoimmune disease, hematologic tumor, or solid tumor;
preferably, the autoimmune disease is selected from: systemic lupus erythematosus (SLE), polymyositis and dermatomyositis, systemic scleroderma, Sjögren's syndrome, autoimmune hemolytic anemia, or rheumatoid arthritis;
preferably, the hematologic tumor or solid tumor is selected from: epithelial cell carcinoma, glioblastoma, lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, lung cancer, digestive tract cancer, bladder cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma;
more preferably, the hematologic tumor or solid tumor is selected from: epithelial cell carcinoma, glioblastoma, rectal cancer, breast cancer, gastric cancer, metastatic brain cancer, metastatic liver cancer, lung cancer, digestive tract cancer, mesothelioma, pancreatic cancer, ovarian cancer, and B-cell malignancies.

In twelfth aspect of the present invention, provided is a method for treating a disease in a subject. The method comprises administering to the subject a therapeutically effective amount of any one of the single-domain antibody as described, any one of the STAR as described, the CAR as described above, the therapeutic immune cells as described above, and/or any one of the expression vector as described and/or the pharmaceutical composition as described above. Preferably, the disease is a CCR8-related disease, such as a CCR8-related autoimmune disease, hematological malignancy, or solid tumor.

Preferably, the autoimmune disease is selected from the following group: systemic lupus erythematosus (SLE), polymyositis and dermatomyositis, systemic scleroderma, Sjögren's syndrome, autoimmune hemolytic anemia, or rheumatoid arthritis.

Preferably, the hematological malignancy or solid tumor is selected from the following group: epithelial cell carcinoma, glioblastoma, lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, lung cancer, digestive tract cancer, bladder cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma.

More preferably, the hematological malignancy or solid tumor is selected from the following group: epithelial cell carcinoma, glioblastoma, rectal cancer, breast cancer, gastric cancer, metastatic brain cancer, metastatic liver cancer, lung cancer, digestive tract cancer, mesothelioma, pancreatic cancer, ovarian cancer, or B-cell malignancies.

### BRIEF DESCRIPTION OF THE DRAWINGS

FIG. 1. Results of the affinity detection of CCR8 nanobody EC50;

FIG. 2. Results of specificity detection of CCR8 nanobody MPA;

FIG. 3. Structural diagrams of different STARs targeting CCR8;

FIG. 4. Comparison results of the in vitro killing effects of CCR8 STAR;

FIG. 5. Tumor fluorescence photos of CCR8 STAR in mouse tumor models;

FIG. 6. Tumor Fluorescence Value Changes of CCR8 STAR in Mouse Tumor Models:

FIG. 7. Tumor Volume Changes of CCR8 STAR in Mouse Tumor Models;

FIG. 8A. In vitro killing effect result of dual-target CCR8-MSLN STAR-T on H9 target cell;

FIG. 8B. In vitro killing effect result of dual-target CCR8-MSLN STAR-T on 293T-hCCR8 target cell;

FIG. 8C. In vitro killing effect result of dual-target CCR8-MSLN STAR-T on 293T target cell;

FIG. 8D. In vitro killing effect result of dual-target CCR8-MSLN STAR-T on 293T-MSLN target cell;

FIG. 9. Tumor fluorescence photos of CCR8-MSLN STAR in mouse tumor models;

FIG. 10. Tumor fluorescence value changes of CCR8-MSLN STAR in mouse tumor models;

FIG. 11. Body weight changes of CCR8-MSLN STAR in mouse tumor models;

FIG. 12. Tumor volume changes of CCR8-MSLN STAR in mouse tumor models;

FIG. 13. Results of detection of the infection efficiencies of CCR8-Claudin18.2 STAR with mbIL-15. The results show similar infection efficiency of different STAR virus structures;

FIG. 14. In vitro killing effect results of Claudin18.2 positive cells by CCR8-Claudin18.2 STAR Co-expressing mbIL-15;

FIG. 15A. In vitro killing effect results of CCR8 positive cells (293T-hCCR8 cell line) by CCR8-Claudin18.2 STAR Co-expressing mbIL-15;

FIG. 15B. In vitro killing effect results of CCR8 positive cells (H9 cell line) by CCR8-Claudin18.2 STAR Co-expressing mbIL-15;

FIG. 16A. For 293T-hClaudin18.2 target cells, cytokine secretion results of CCR8-Claudin18.2 STAR co-expressing mbIL-15;

FIG. 16B. For 293T-hCCR8 target cells, cytokine secretion results of CCR8-Claudin18.2 STAR co-expressing mbIL-15;

FIG. 17. Tumor fluorescence photos of CCR8-Claudin18.2 STAR co-expressing mbIL-15 in mouse tumor models;

FIG. 18. Body weight changes of CCR8-Claudin18.2 STAR co-expressing mbIL-15 in mouse tumor models;

FIG. 19. Tumor fluorescence value changes of CCR8-Claudin18.2 STAR co-expressing mbIL-15 in mouse tumor models;

FIG. 20. Tumor volume changes of CCR8-Claudin18.2 STAR co-expressing mbIL-15 in mouse tumor models;

FIG. 21. Tumor volume inhibition rate changes of CCR8-Claudin18.2 STAR co-expressing mbIL-15 in mouse tumor models;

FIG. 22. Schematic diagram of the structures of CCR8 and dual-target CCR8-MSLN CAR-T;

FIG. 23. Results of detection of the infection efficiencies of CARs with Different Targets;

FIG. 24A. For 293T cells, results of the in vitro killing effects of CCR8-MSLN STAR;

FIG. 24B. For 293T target cells overexpressing hCCR8, results of the in vitro killing effects of CCR8-MSLN STAR;

FIG. 24C. For 293T target cells overexpressing hMSLN, results of the in vitro killing effects of CCR8-MSLN STAR;

FIG. 25. Schematic diagram of the structures of CCR8 and dual-target CCR8-Claudin18.2 CAR-T;

FIG. 26. Results of detection of the infection efficiencies of CARs with Different Targets:

FIG. 27A. For the 293T target cells, results of the in vitro killing effects of CCR8-Claudin18.2 CAR-T;

FIG. 27B. For the 293T target cells overexpressing hCCR8, results of the in vitro killing effects of CCR8-Claudin18.2 CAR-T;

FIG. 27C. For the 293T target cells overexpressing hClaudin18.2, results of the in vitro killing effects of CCR8-Claudin18.2 CAR-T.

### DETAILED DESCRIPTION

### Definitions

Unless otherwise indicated or defined, all terms used have their ordinary meanings in the art, which will be understood by those skilled in the art. Reference is also made, for example, to standard manuals such as Sambrook et al. “Molecular Cloning: A Laboratory Manual” (2nd Edition), Volumes 1-3, Cold Spring Harbor Laboratory Press (1989); Lewin, “Genes IV,” Oxford University Press, New York (1990); and Roitt et al., “Immunology” (2nd Edition), Gower Medical Publishing, London, New York (1989), as well as the general prior art cited herein. Moreover, unless otherwise indicated, all methods, steps, techniques and operations not specifically described may be, and have been carried out in manners known per se, and the manners will be known by those skilled in the art. Reference is also made, for example, to standard manuals, the above general prior art and other references cited therein.

As used herein, the term “and/or” covers all combinations of items linked by the term, and it should be considered that each combination has been individually listed herein. For example, “A and/or B” covers “A”, “A and B” and “B”. For example, “A, B and/or C” covers “A”, “B”, “C”, “A and B”, “A and C”, “B and C” and “A and B and C”.

When the term of “comprise” or “include” or “contain” is used herein to describe a sequence of a protein or nucleic acid, the protein or nucleic acid may consist of the sequence, or one or both ends of the protein or nucleic acid may have additional amino acids or nucleotides, but the activity described in the present invention still exists. Furthermore, it is clear to those skilled in the art that methionine encoded by an initiation codon at the N-terminal of a polypeptide will be retained under certain practical circumstances (for example, when expressed in a particular expression system), but the function of the polypeptide is not substantially affected. Therefore, when a particular polypeptide amino acid sequence is described in the specification and claims of the present invention, although the methionine encoded by the initiation codon at the N-terminal may not be contained, a sequence comprising the methionine is also covered in this regard, and accordingly, an encoding nucleotide sequence may also comprise the initiation codon; and vice versa.

The term “isolated” refers to a polypeptide or nucleic acid molecule that is considered “isolated” when it has been separated from at least one other component (e.g., another protein/polypeptide, another nucleic acid, another biological component or macromolecule, or at least one contaminant, impurity, or trace component) that is typically associated with it in its natural biological source and/or the reaction medium or culture medium from which the polypeptide or nucleic acid molecule it is obtained. Specifically, a polypeptide or nucleic acid molecule is considered “isolated” when it has been purified at least 2-fold, particularly at least 10-fold, more particularly at least 100-fold, and up to 1000-fold or more. As determined by suitable techniques (e.g., appropriate chromatographic techniques such as polyacrylamide gel electrophoresis), the “isolated” polypeptide or nucleic acid molecule is preferably substantially homogeneous.

As used herein, “Synthetic T-cell Receptor and Antigen Receptor (STAR)” refers to a modified TCR in which the variable regions of the TCR are replaced with antibody variable regions or other receptor sequences, and the constant regions may also be modified.

As used herein, the “antigen-binding region” (e.g., the antigen-binding region in STAR) means that it can specifically bind to the target antigen alone or in combination with another antigen-binding region. The antigen-binding region may be derived from an antibody that specifically binds to the target antigen, including any commercially available antibody. The antigen-binding region may also be derived from a receptor that binds to a specific target protein.

As used herein, the term “antibody” refers to immunoglobulins and immunoglobulin fragments, whether naturally or partially or fully synthetically produced (e.g., recombinant), including any fragment that retains the binding specificity of full-length immunoglobulin, including at least a portion of its variable region containing the immunoglobulin molecule. Therefore, antibodies include any protein having a binding domain homologous or substantially homologous to the antigen-binding region (antibody-binding site) of an immunoglobulin. Antibodies include antibody fragments. As used herein, the term antibody includes synthetic antibodies, recombinantly produced antibodies, multispecific antibodies (e.g., bispecific antibodies), human antibodies, non-human antibodies, camelid antibodies, single-domain antibodies, humanized antibodies, chimeric antibodies, intracellular antibodies, and antibody fragments, including but not limited to Fab fragments, Fab′ fragments, F(ab′)₂ fragments, Fv fragments, disulfide-linked Fv (dsFv), Fd fragments, Fd′ fragments, single-chain Fv (scFv), single-chain Fab (scFab), diabodies, anti-idiotypic (anti-Id) antibodies, or antigen-binding fragments of any of the foregoing antibodies. The antibodies described herein include any immunoglobulin type (e.g., IgG, IgM, IgD, IgE, IgA, and IgY), any class (e.g., IgG1, IgG2, IgG3, IgG4, IgA1, and IgA2), or subclass (e.g., IgG2a and IgG2b).

As used herein, the variable domain or variable region is a specific Ig domain of the heavy or light chain of an antibody, that contain amino acid sequences that vary between different antibodies. Each light chain and each heavy chain has a variable region VL (also denoted as VL) and VH (or also denoted as VH), respectively. The variable domains provide antigen specificity and are therefore responsible for antigen recognition. Each variable region contains CDRs and framework regions (FRs), with CDRs being part of the antigen-binding site.

As used herein, “hypervariable region,” “HV,” “complementarity-determining region,” “CDR,” and “antibody CDR” are used interchangeably to refer to one of several segments within each variable region that together form the antigen-binding site of an antibody. Each variable region contains 3 CDRs, designated CDR1, CDR2, and CDR3. For example, for a conventional 4-chain antibody, the light chain variable region contains 3 CDRs, designated VL CDR1, VL CDR2, and VL CDR3 (or LCDR1, LCDR2, and LCDR3); the heavy chain variable region domain contains 3 CDRs, designated VH CDR1, VH CDR2, and VH CDR3 (or HCDR1, HCDR2, and HCDR3). For camelid antibodies or single-domain antibodies, since they have only one variable region, they contain only 3 CDRs, designated CDR1, CDR2, and CDR3.

In the context of the present invention, the terms “single-domain antibody,” “nanobody,” “heavy-chain single-domain antibody,” “VHH,” “VHH domain,” “VHH antibody fragment,” and “VHH antibody” are used interchangeably.

A “single-domain antibody” is the variable domain of an antigen-binding immunoglobulin known as a “heavy-chain antibody” (i.e., an “antibody lacking light chains”) (Hamers-Casterman C, Atarhouch T, Muyldermans S, Robinson G, Hamers C, Songa E B, Bendahman N, Hamers R.: “Naturally occurring antibodies devoid of light chains”; Nature 363,446-448 (1993)). The term “VHH” is used to distinguish the variable region of heavy-chain antibodies from the heavy-chain variable region (denoted as “VH” herein) present in conventional four-chain antibodies and the light-chain variable region (denoted as “VL” herein) present in conventional four-chain antibodies. VHH specifically binds to epitopes without the need for other antigen-binding regions (unlike VH or VL in conventional 4-chain antibodies, where epitopes are recognized by VL and VH together). VHH is a small, stable, and efficient antigen recognition unit formed by a single domain.

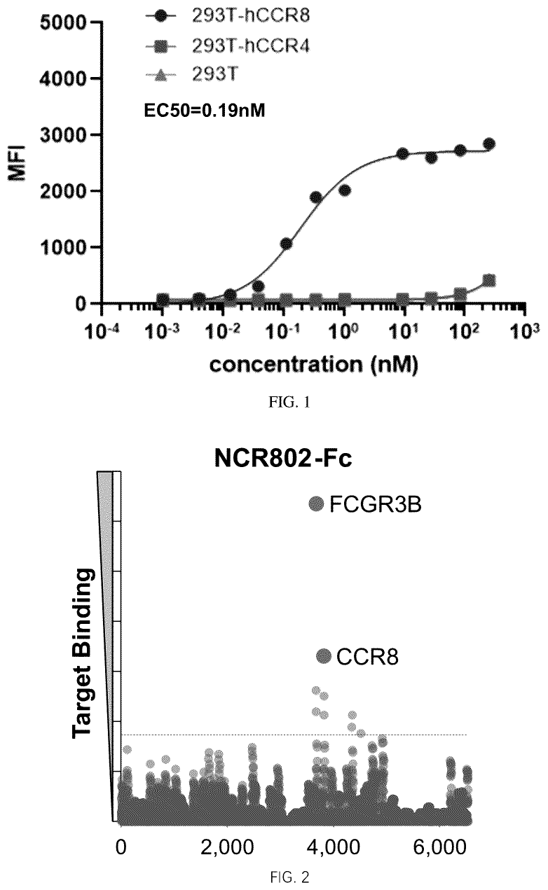

For example, as shown in FIG. 2 of Riechmann and Muyldermans, J. Immunol. Methods 231,25-38 (1999), the amino acid residues of the VHH domain of camelids can be numbered according to the general numbering scheme for VH domains provided by Kabat et al. (Kabat et al., Sequences of Proteins of Immunological Interest, 5th Ed. Public Health Service, National Institutes of Health, Bethesda, Md. (1991)). However, alternative methods for numbering the amino acid residues of VH domains are known in the art and can also be similarly applied to VHH domains. For example, Chothia CDRs refer to the location of structural loops (Chothia and Lesk, J. Mol. Biol. 196:901-917 (1987)). The AbM CDR represents a compromise between Kabat hypervariable regions and the Chothia structural loops and is used in Oxford Molecular's AbM antibody modeling software. The “Contact” CDRs is based on the analysis of available complex crystal structures. The CDRs of the single-domain antibodies of the present invention are determined according to the Kabat database.

VHH domains derived from camelids can be “humanized” (also referred to herein as “sequence optimization”, in addition to humanization, “sequence optimization” may also encompass other modifications to the sequence by providing one or more mutations that improve the properties of VHH, such as removing potential post-translational modification sites) by replacing one or more amino acid residues in the original VHH sequence with one or more amino acid residues present at corresponding positions in the VH domain of human conventional four-chain antibodies. Humanized VHH domains may contain one or more fully human framework region sequences. Humanization can be achieved using methods such as protein surface resurfacing and/or CDR grafting to a universal human framework.

In general, the term “specificity” refers to the number of different types of antigens or epitopes that a particular antigen-binding molecule or antigen-binding protein (e.g., the antibody of the present invention) can bind. The specificity of an antigen-binding protein can be determined based on its affinity and/or avidity. The affinity, expressed as the dissociation equilibrium constant (KD) of the antigen with the antigen-binding protein, is a measure of the binding strength between the epitope and the antigen-binding site on the antigen-binding protein: the smaller the KD value, the stronger the binding between the epitope and the antigen-binding protein (or, affinity can also be expressed as the association constant (KA), which is 1/KD). As will be understood by those skilled in the art, depending on the specific antigen of interest, affinity can be determined in a known manner. Avidity is a measure of the binding strength between an antigen-binding protein (e.g., an antibody) and its related antigen. Avidity is related to both the affinity between the antigen and the antigen-binding site on the antigen-binding protein and the number of related binding sites present on the antigen-binding protein.

As used herein, “the amino acid number refers to SEQ ID NO: x” (SEQ ID NO: x is a specific sequence listed herein) means that the position number of the specific amino acid described is the position number of the corresponding amino acid on SEQ ID NO: x. The correspondence of amino acids in different sequences can be determined according to sequence alignment methods well known in the art. For example, the correspondence of the amino acids can be determined by an online alignment tool from EMBL-EBI (https://www.ebi.ac.uk/Tools/psa/), wherein two sequences can be aligned by using a Needleman-Wunsch algorithm by using default parameters. For example, an alanine at position 46 from its N-terminal of a polypeptide aligns with the amino acid at position 48 of SEQ ID NO: x in a sequence alignment, then the amino acid in the polypeptide may also be described herein as “an alanine at position 48 of the polypeptide, and the amino acid position refers to SEQ ID NO: x”.

The proteins/polypeptides mentioned in the present invention may contain a signal peptide (or leader sequence) at the N-terminal. Those skilled in the art will understand that in cells, the signal peptide sequence can direct the protein/polypeptide to a specific location in the cell, such as the cell membrane, and it may be cleaved and not included in the final product. Exemplary signal peptides include, but are not limited to, the IgE signal peptide (SEQ ID NO:37), GM-CSF signal peptide, bovine prolactin pre-signal peptide, etc. These signal peptide sequences are known in the art or can be easily identified by those skilled in the art based on the existing knowledge in the field.

The “expression vector” of the present invention may be a linear nucleic acid fragment, a cyclic plasmid, a viral vector, or an RNA capable of translation (e.g., mRNA). In some preferred embodiments, the expression vector is a viral vector, such as a lentiviral vector.

As used herein, the term “operably linked” means that a regulatory element (e.g., but not limited to, a promoter sequence, a transcription termination sequence, etc.) is linked to a nucleic acid sequence (e.g., a coding sequence or an open reading frame) such that the nucleotide sequence transcription is controlled and regulated by the transcriptional regulatory element. Techniques for operably linking a regulatory element region to a nucleic acid molecule are known in the art.

The term “regulatory sequence” and “regulatory element” are used interchangeably to refer to a nucleotide sequence that is located upstream (5′ non-coding sequence), intermediate or downstream (3′ non-coding sequence) of a coding sequence and affect the transcription, RNA processing or stability or translation of the relevant coding sequence. An expression regulatory element refers to a nucleotide sequence that can control the transcription, RNA processing or stability, or translation of a nucleotide sequence of interest. A regulatory sequence may include, but is not limited to, a promoter, a translation leader sequence, an intron, an enhancer, and a polyadenylation recognition sequence. Suitable promoters include, but are not limited to, the PGK promoter, hEF1a HTLV promoter (as shown in SEQ ID NO:36), and MND promoter (SEQ ID NO: 35).

As use herein, “subject” refers to an organism that suffers from or is prone to suffer from a disease (e.g., cancer) that can be treated by the antibody, the cell, method, or pharmaceutical composition of the present invention. A non-limiting example includes human, cattle, rat, mouse, dog, monkey, goat, sheep, cow, deer, and other non-mammals. In some preferred embodiments, the subject is human.

As used herein, a “pharmaceutically acceptable carrier” includes any and all physiologically compatible solvents, dispersion medium, coatings, antibacterial and antifungal agents, isotonic agents and absorption retarders, etc. Preferably, the carrier is suitable for intravenous, intramuscular, subcutaneous, parenteral, spinal, or epidermal administration (e.g., by injection or infusion).

As used herein, a “therapeutically effective amount” or “therapeutically effective dose” or “effective amount” refers to the amount of a substance, compound, material or cell that is at least sufficient to produce a therapeutic effect after administration to a subject. Therefore, it is an amount necessary to prevent, cure, improve, block or partially block the symptoms of disease or disorder. For example, an “effective amount” of the cell or pharmaceutical composition of the present invention may preferably result in a decrease in the severity of disorder symptoms, an increase in the frequency and duration of the asymptomatic period of the disorder, or the prevention of injury or disability as a result of suffering from the disorder. For example, for the treatment of tumor, an “effective amount” of the antibody, the cell, the expression vector or pharmaceutical composition of the present invention may preferably inhibit tumor cell growth or tumor growth by at least about 10%, preferably at least about 20%, more preferably at least about 30%, more preferably at least about 40%, more preferably at least about 50%, more preferably at least about 60%, more preferably at least about 70%, and more preferably at least about 80%, as compared to an untreated subject. The ability to inhibit tumor growth can be evaluated in an animal model system that may predict efficacy in a human tumor. Alternatively, it is possible to perform evaluation by examining the ability to inhibit the growth of tumor cells which may be determined in vitro by tests known to those skilled in the art.

### Single-Domain Antibody Specifically Binding to CCR8

In one aspect, the present invention provides a single-domain antibody that specifically binds to CCR8, comprising CDR1, CDR2, and CDR3 selected from CDR1, CDR2 and CDR3 in either SEQ ID NO:4 or 8. The CDRs can be Kabat CDRs, AbM CDRs, Chothia CDRs, or Contact CDRs. In some embodiments, the CDRs are Kabat CDRs.

In some embodiments, for the convenience of purification and/or labeling, the single-domain antibody that specifically binds to CCR8 may further comprise one or more additional tag sequences. For example, the additional tag can be a His-tag (such as a 6×His-tag) or an Fc-tag, which is beneficial for the separation and purification of the polypeptide or for prolonging its in-vivo half-life. Those skilled in the art will understand that these additional tags will not substantially affect the binding ability of the antibody.

The single-domain antibody specifically binding to CCR8 provided by the present invention can have a KD value for binding to CCR8 of less than about 1×10⁻⁷ M, preferably less than about 1×10⁻⁸ M, more preferably less than about 1×10⁻⁹ M, and even more preferably less than about 1×10⁻¹⁰ M.

### Expression Vectors and Methods for Preparing Single-Domain Antibodies

In another aspect, the present invention provides an isolated nucleic acid molecule encoding the single-domain antibody that specifically binds to CCR8 of the present invention. In some embodiments, the nucleotide sequence of the nucleic acid molecule is codon-optimized for the host cell used for expression. In some embodiments, the nucleic acid molecule of the present invention is operably linked to an expression regulatory element such as a promoter.

The present invention also provides an expression vector for expressing the single-domain antibody of the present invention, which contains the nucleic acid molecule encoding the single-domain antibody that specifically binds to CCR8 as described herein.

The present invention also provides a host cell for producing the single-domain antibody of the present invention, which is transformed with the aforementioned nucleic acid molecule or expression vector. As used herein, a “host cell” is a cell used to receive, maintain, replicate, and amplify a vector. Host cells can also be used to express the polypeptide encoded by the nucleic acid or vector. When the host cell divides, the nucleic acid contained in the vector is replicated, thereby amplifying the nucleic acid. Host cells can be eukaryotic cells or prokaryotic cells. Suitable host cells include, but are not limited to, CHO cells, various COS cells, HeLa cells, HEK cells such as HEK 293 cells.

In another aspect, the present invention provides a method for producing the single-domain antibody that specifically binds to CCR8 as described herein, comprising:
(i) Culturing the host cell of the present invention under conditions suitable for the expression of the nucleic acid molecule or expression vector, and
(ii) Isolating and purifying the single-domain antibody that specifically binds to CCR8 expressed by the host cell.

The methods and reagents for recombinantly producing polypeptides, such as specific suitable expression vectors, transformation or transfection methods, selection markers, methods for inducing protein expression, culture conditions, etc., as well known in the art. Similarly, the protein separation and purification techniques applicable to the method for producing the single-domain antibody that specifically binds to CCR8 of the present invention are well-known to those skilled in the art.

However, the single-domain antibody that specifically binds to CCR8 of the present invention can also be obtained by other methods known in the art for producing proteins, such as chemical synthesis, including solid-phase or liquid-phase synthesis.

### STAR Targeting CCR8

In some aspects, the present invention provides a synthetic T-cell receptor and antigen receptor (STAR) targeting CCR8, which contains an antigen-binding region that specifically binds to CCR8.

In some embodiments, the synthetic T-cell receptor and antigen receptor (STAR) targeting CCR8 comprises a first peptide chain and a second peptide chain, wherein the first peptide chain contains a first constant region, the second peptide chain contains a second constant region, and the first peptide chain and/or the second peptide chain further contains an antigen-binding region that specifically binds to CCR8. The first or second constant region is selected from the constant region of the TCRα chain or the TCRβ chain, respectively.

As used herein, “exogenous” means a protein or nucleic acid sequence from a foreign species, or if from the same species, a protein or nucleic acid sequence that has been significantly altered in composition and/or location from its natural form through deliberate human intervention.

As used herein, the “exogenous intracellular functional domain” can be the endodomain of a co-stimulatory molecule such as CD40, OX40, ICOS, CD28, 4-1BB, CD27, CD137; it can also be the endodomain of a co-inhibitory molecule, such as TIM3, PD1, CTLA4, LAG3; it can also be the endodomain of a cytokine receptor such as an interleukin receptor (such as the IL-2β receptor, IL-7α receptor, or IL-21 receptor), an interferon receptor, a tumor necrosis factor superfamily receptor, a colony-stimulating factor receptor, a chemokine receptor, a growth factor receptor, or other membrane-protein: or it can be the domain of an intracellular protein such as NIK.

In some preferred embodiments, the exogenous intracellular functional domain is the endodomain of a co-stimulatory molecule, preferably the endodomain of OX40. In some embodiments, the endodomain of OX40 comprises the amino acid sequence shown in SEQ ID NO: 23.

The target-binding region refers to the region in an antigen receptor (such as STAR or CAR) that is used to specifically bind to a target antigen. The target-binding region can comprise one or more antigen-binding regions. The antigen-binding regions bind to a single protein region or epitope (antigenic determinant) of an antigen through antigen-antibody interactions or receptor-ligand interactions. In some embodiments, the antigen-binding region is an antibody or an antigen-binding fragment of an antibody, such as a single-domain antibody, scFv, or nanobody, etc.

### Chimeric Antigen Receptor (CAR) Targeting CCR8

In some aspects, the present invention provides a chimeric antigen receptor (CAR) targeting CCR8, which contains an extracellular antigen-binding region (antigen-binding domain). The extracellular antigen-binding domain contains a CCR8 single-domain antibody. the CAR, from the N-terminal to the C-terminal, sequentially comprises an extracellular antigen-binding region, a hinge region, a transmembrane domain, a co-stimulatory domain, and an intracellular signal-transduction domain.

The hinge region is the region in a chimeric antigen receptor (CAR) that connects the extracellular antigen-binding region and the transmembrane domain. The hinge region is usually composed of a flexible peptide chain, which is used to provide spatial freedom and enhance the efficiency of antigenbinding.

The transmembrane domain is the region in a chimeric antigen receptor (CAR) that anchors the CAR in the cell membrane. The transmembrane domain is usually composed of hydrophobic amino acids which can stably embed in the cell membrane.

The intracellular signal-transduction domain is the region in a chimeric antigen receptor (CAR) that is used to transmit activation signals. It usually contains immunoreceptor tyrosine-based activation motifs (ITAM) or other signaling motifs. These domains can activate the immune response of T cells.

In some embodiments, the CAR further comprises a transmembrane domain, such as the CD8a transmembrane domain or the CD28 transmembrane domain, preferably the CD8a transmembrane domain.

In some embodiments, the CAR further includes a hinge region located between the extracellular antigen-binding region and the transmembrane domain. For example, the hinge region is the CD8a hinge region.

In some embodiments, the CAR further contains a signal-transduction domain, such as a signal-transduction domain that can be used for T-cell activation, for example, the signal-transduction domains selected from signal-transduction domains of TCRζ, FcRγ, FcRβ, FcRε, CD3γ, CD3δ, CD3ε, CD3ζ, CD5, CD22, CD79a, CD79b, and CD66d. In some preferred embodiments, the CAR contains the CD3ζ signal-transduction domain.

In some embodiments, the CAR further contains one or more co-stimulatory domains, such as the co-stimulatory domains selected from co-stimulatory domains of CD3, CD27, CD28, CD83, CD86, CD127, 4-1BB, and 4-1BBL.

In some embodiments, the CAR, in the direction from the N-terminus to the C-terminus, contains the extracellular antigen-binding region, the hinge region, the transmembrane domain, the co-stimulatory domain, and the signal-transduction domain. In some embodiments, the hinge region is the CD8α hinge region, the transmembrane domain is the CD8α transmembrane domain, the signal-transduction domain is the CD3ζ signal-transduction domain, and the co-stimulatory domain is the 4-1BB co-stimulatory domain.

In some embodiments, the extracellular antigen-binding region further contains an antigen-binding region that specifically binds to another antigen. Thus, the CAR can also target the other antigen. Preferably, the antigen-binding region that specifically binds to the other antigen comprises a single-chain antibody (scFv) or a single-domain antibody that specifically binds to the other antigen.

In some embodiments, the other antigen is MSLN, wherein the antigen-binding region specifically binding to MSLN comprises CDR1 as shown in SEQ ID NO:31, CDR2 as shown in SEQ ID NO:32, and CDR3 as shown in SEQ ID NO:33. In some embodiments, the other antigen is MSLN, wherein the antigen-binding region specifically binding to MSLN comprises the amino acid sequence shown in SEQ ID NO:34 (single-domain antibody).

In some embodiments, the other antigen is Claudin18.2, wherein the antigen-binding region specifically binding to Claudin18.2 comprises CDR1 as shown in SEQ ID NO:41, CDR2 as shown in SEQ ID NO:42, and CDR3 as shown in SEQ ID NO:43. In some embodiments, the other antigen is Claudin18.2, wherein the antigen-binding region specifically binding to Claudin18.2 comprises the amino acid sequence shown in SEQ ID NO:44 (single-domain antibody).

In some specific embodiments, the CAR comprises an amino acid sequence shown in any one of SEQ ID NO:59 to 64.

### Therapeutic Immune Cells

In another aspect, the present invention provides an isolated therapeutic immune cell, which comprises the STAR of the present invention.

In some embodiments, the immune cell is a T cell. In other embodiments, the immune cell is a natural killer (NK) cell.

In some embodiments, the STAR of the present invention is co-expressed with a membrane-bound IL-15 protein (mbIL-15) in the therapeutic immune cell.

mbIL-15 refers to a fusion protein formed by the connection (such as through a linker) of IL-15 and the extracellular domain of IL-15Ra. The exemplary amino acid sequence of IL-15 is shown in SEQ ID NO:27, but also includes amino acid sequences having at least 80%, at least 85%, at least 90%, at least 95%, and even at least 99% sequence identity with SEQ ID NO:27. The exemplary amino acid sequence of the extracellular domain of IL-15Rα is shown in SEQ ID NO: 28, but also includes amino acid sequences having at least 80%, at least 85%, at least 90%, at least 95%, and even at least 99% sequence identity with SEQ ID NO:28. The exemplary amino acid sequence of the linker connecting the extracellular domain of IL-15Ra and IL-15 is shown in SEQ ID NO:29. The exemplary amino acid sequence of mbIL-15 is shown in SEQ ID NO:30, but also includes amino acid sequences having at least 80%, at least 85%, at least 90%, at least 95%, and even at least 99% sequence identity with SEQ ID NO:30.

The immune cells of the present invention, such as T cells, can be obtained from many non-limiting sources through various non-limiting methods, including peripheral blood mononuclear cells, bone marrow, lymph node tissue, umbilical cord blood, thymus tissue, ascites, pleural effusion, spleen tissue, and tumors. In some embodiments, the cells can be derived from healthy donors or patients diagnosed with cancer. In some embodiments, the cells can be part of a mixed population of cells with different phenotypic characteristics. For example, immune cells such as T cells can be obtained by isolating peripheral blood mononuclear cells (PBMC), followed by activation and expansion with specific antibodies.

In some embodiments, the immune cells such as T cells of the present invention are isolated (ex vivo) immune cells.

In some embodiments of various aspects of the present invention, the immune cells such as T cells are derived from autologous cells of a subject. As used herein, “autologous” means that the cells, cell lines, or cell populations used for treating a subject are derived from the subject. In some embodiments, the immune cells such as T cells are derived from allogeneic cells, for example, from a donor who is human leukocyte antigen (HLA)-compatible with the subject. Cells from a donor can be transformed into non-alloreactive cells using standard protocols and replicated as needed to obtain cells that can be administered to one or more patients.

In some embodiments, the therapeutic immune cells such as T cells are therapeutic immune cells such as T cells that can be obtained or have been obtained by the expression vectors or methods of the present invention described below.

### Expression Vectors and Methods for Preparing Therapeutic Immune Cells

In one aspect, the present invention provides an expression vector comprising the coding sequence of the STAR of the present invention.

The coding sequence in the expression vector of the present invention can be operably linked to regulatory elements such as a promoter for expression in cells.

In some embodiments, the expression vector further comprises the coding sequence of the membrane-bound IL-15 protein (mbIL-15) of the present invention.

The coding sequence in the expression vector of the present invention can be operably linked to regulatory elements such as a promoter for expression in cells.

In some embodiments, the mbIL-15 can be driven by a separate promoter for expression.

In some embodiments, the expression vector comprises:
a) A coding nucleotide sequence of a fusion polypeptide in which the first peptide chain of the STAR of the present invention and the second peptide chain of the STAR of the present invention are linked by a self-cleaving peptide;
b) A coding nucleotide sequence of a fusion polypeptide in which the first peptide chain of the STAR of the present invention, the second peptide chain of the STAR of the present invention, and the mbIL-15 of the present invention are linked by a self-leaving peptide; or
c) A coding nucleotide sequence of a fusion polypeptide in which the mbIL-15 of the present invention is linked by a self-cleaving peptide.

In some embodiments, the expression vector comprises a coding nucleotide sequence of a fusion polypeptide in which the first peptide chain of the STAR of the present invention and the second peptide chain of the STAR of the present invention are linked by a self-cleaving peptide.

As used herein, the “self-cleavage peptide” means a peptide that can achieve self-cleavage in a cell. For example, the self-cleavage peptide may contain a protease recognition site so as to be recognized and specifically cleaved by proteases in a cell. Alternatively, the self-cleavage peptide may be a 2A polypeptide. The 2A polypeptide is a kind of short peptide from virus, and its self-cleavage occurs during translation. When two different target proteins are linked by 2A polypeptide and expressed in the same reading frame, the two target proteins are generated almost in a ratio of 1:1. A common 2A polypeptide may be P2A from porcine techovirus-1, T2A from Thosea asigna virus, E2A from equine rhinitis A virus, and F2A from foot-and-mouth disease virus. Among them, P2A has the highest cleavage efficiency and is therefore preferred. A variety of functional variants of these 2A polypeptides are also known in the art, which can also be used in the present invention. 2A polypeptides can also be combined with a Furin recognition sequence to remove additional introduced amino acid sequences.

In some embodiments, the self-cleaving peptide is a 2A polypeptide, such as the P2A polypeptide. In some embodiments, the self-cleaving peptide is a Furin-2A polypeptide, such as the Furin-P2A polypeptide shown in SEQ ID NO:26.

In some embodiments, the different parts of the fusion polypeptide can be arranged in different ways as long as they are separated by a self-cleaving peptide. For example, in some embodiments, the fusion polypeptide can comprise, in the direction from the N-terminal to the C-terminal, the second peptide chain (the peptide chain containing the constant region of the TCRβ chain), a self-cleaving peptide such as Furin-P2A, and the first peptide chain (the peptide chain containing the constant region of the TCRα chain). When there are multiple self-cleaving peptides, the self-cleaving peptides can be the same or different.

In another aspect, the present invention provides a method for preparing therapeutic immune cells, comprising:
Step 1) Providing starting immune cells;
Step 2) Introducing the expression vector of the present invention into the starting immune cells; and
Step 3) Harvesting the immune cells obtained in Step 2).

In some embodiments, the starting immune cells are T cells. In other embodiments, the starting immune cells are NK cells.

The starting immune cells of the present invention, such as T cells, can be obtained from many non-limiting sources through various non-limiting methods, including peripheral blood mononuclear cells, bone marrow, lymph node tissue, umbilical cord blood, thymus tissue, ascites, pleural effusion, spleen tissue, and tumors. In some embodiments, the cells can be derived from healthy donors or patients diagnosed with cancer. In some embodiments, the cells can be part of a mixed population of cells with different phenotypic characteristics. For example, starting immune cells such as T cells can be obtained by isolating peripheral blood mononuclear cells (PBMCs) and then activating and expanding them with specific antibodies.

In some embodiments, the starting immune cells, such as T cells, are isolated (ex-vivo) immune cells, such as T cells. Therefore, the therapeutic immune cells, such as T cells, obtained by the present invention are also isolated (ex-vivo) therapeutic immune cells, such as T cells.

In some embodiments, the method of the present invention is an in-vitro method.

In some embodiments of various aspects of the present invention, the starting immune cells, such as T cells, are derived from the autologous cells of a subject. As used herein, “autologous” means that the cells, cell lines, or cell populations used for treating a subject are derived from the subject. In some embodiments, the starting immune cells, such as T cells, are derived from allogeneic cells, for example, from a donor who is human leukocyte antigen (HLA)-compatible with the subject. Standard protocols can be used to convert the cells from the donor into non-alloreactive cells and be replicated them as needed to produce cells that can be administered to one or more patients.

The introduction of the expression vector into immune cells, such as T cells, can be performed using by methods known in the art, including but not limited to microinjection, electroporation, virus-mediated transfection, liposome-mediated transfection, etc.

In some embodiments, the method further includes, between Step 2) and Step 3), Step x: Expand the immune cells, such as T cells, obtained in Step 2). Immune cells, such as T cells, may be expanded using methods known in the art.

In some embodiments, the method further comprises Step y: Screen for immune cells, such as T cells, that express the STAR. In some embodiments, Step y) can be performed after Step 2). In some embodiments, Step y) can be carried out after Step 2) and before Step x). In some embodiments, Step y) can be performed after Step x). In some embodiments, the screening is performed by flow cytometry.

In another aspect, the present invention provides therapeutic immune cells, such as T cells, that can be obtained or are obtained by the expression vector or the method of the present invention.

### Pharmaceutical Compositions and Applications

In another aspect, the present invention provides a pharmaceutical composition, which comprises the single-domain antibody of the present invention, the therapeutic immune cells of the present invention, and/or the expression vector of the present invention, and a pharmaceutically acceptable carrier.

In another aspect, the present invention provides the use of the single-domain antibody of the present invention, the therapeutic immune cells of the present invention, the expression vector of the present invention, and/or the pharmaceutical composition of the present invention in the preparation of a medicament for treating a disease in a subject.

In another aspect, the present invention provides a method for treating a disease in a subject. which includes administering to the subject a therapeutically effective amount of the single-domain antibody of the present invention, the therapeutic immune cells of the present invention, the expression vector of the present invention, and/or the pharmaceutical composition of the present invention.

In practical applications, the dosage levels of the antibody, cells, or expression vector in the pharmaceutical composition of the present invention may vary to obtain the amount of active ingredient that can achieve the desired therapeutic response for a specific patient, the composition, and the mode of administration, without toxicity to patients. The selected dosage level depends on various pharmacokinetic factors, including the activity of the specific composition of the present invention, the route of administration, the time of administration, the excretion rate of the specific compound used, the duration of treatment, other drugs, compounds, and/or materials used in combination with the specific composition, the age, gender, weight, condition, general health, and medical history of the patient being treated, as well as other similar factors well-known in the medical field.

The antibody, expression vector, therapeutic immune cells, pharmaceutical composition, or medicament according to the present invention can be administered in any convenient way, including injection, infusion, implantation, or transplantation. The antibody, expression vector, therapeutic immune cells, or pharmaceutical composition described herein can be administered via intravenous, intralymphatic, intradermal, intratumoral, intramedullary, intramuscular, or intraperitoneal routes. In one embodiment, the antibody, expression vector, therapeutic immune cells, or pharmaceutical composition of the present invention is preferably administered by intravenous injection.

In the embodiments of various aspects of the present invention, the disease is a CCR8-related disease, such as a diseases related to abnormal expression of CCR8, such as a CCR8-related autoimmune disease, hematologic tumor, or solid tumor;

preferably, wherein the autoimmune disease is selected from the following group: systemic lupus erythematosus (SLE), polymyositis, systemic scleroderma, Sjögren's syndrome, autoimmune hemolytic anemia, or rheumatoid arthritis;

Preferably, the tumor is selected from the following group: lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, lung cancer, digestive tract cancer, bladder cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, or sarcoma.

More preferably, the tumor is selected from the following group: rectal cancer, breast cancer, gastric cancer, metastatic brain cancer, metastatic liver cancer, lung cancer, digestive tract cancer, mesothelioma, pancreatic cancer, ovarian cancer, and B-cell malignancies.

### Advantages of the Technical Solution

The present invention provides a cell therapy product that simultaneously targets tumor antigens and receptors in immunosuppressive cells. Its advantage lies in that while killing tumor cells, it specifically targets Treg cells infiltrating the tumor tissue, which can alleviate the suppressive effects of Tregs, improve the tumor immunosuppressive microenvironment and systemic immunity, enhance the in-vivo persistence and function of immune cell products, and improve the clinical efficacy of existing cell immunotherapies.

(1) The dual-functional STAR-T cells of the present invention combine the advantages of both CAR-T and TCR-T cells. The dual-functional STAR-T cells have the high affinity comparable to CAR-T cells and the high signal-transduction capacity and high sensitivity to antigens similar to TCR-T cells. Meanwhile, The dual-functional STAR-T cells avoid the limitations of both CAR-T and TCR-T cells, avoiding the HLA dependence of TCR-T cells and avoiding the relatively serious self-activation of CAR-T cells, thereby reducing the likelihood of exhaustion.

(2) The dual-functional STAR-T cells of the present invention fully utilize their natural multi-chain structure. By attaching an antibody portion targeting a receptor (such as CCR8) on immunosuppressive immune cells to one chain and an antibody portion targeting tumor antigens to the other chain, they achieve dual functions. Due to the structural advantage, the two antibody fragments do not interfere with each other and can function independently. Moreover, the antibody fragment targeting the receptor on immunosuppressive immune cells can eliminate the immunosuppressive effect of Treg cells and improve the tumor immunosuppressive microenvironment, thereby enhancing the tumor-killing ability of the antibody fragment targeting tumor antigens.

(3) In animal models of epithelial cell carcinoma, glioblastoma, and liver cancer, the dual-functional STAR-T cells of the present invention demonstrate superior anti-tumor effects compared to traditional CAR-T cells, with no significant toxic side effects.

(4) The present invention further optimizes the STAR structure by incorporating the co-stimulatory molecule OX40. Cell-based experiments show that the dual-functional STAR-T cells, with OX40 linked to their intracellular domain, exhibit significantly enhanced IL-2 secretion and proliferation levels upon stimulation by target cells.

(5) In the existing technologies, in order to kill tumors while improving the tumor immunosuppressive microenvironment, patients need to receive separate treatments or take different medications. This not only affects the therapeutic effect of tumor treatment, increases the burden on patients, but also raises the risk of adverse drug reactions. The dual-functional STAR-T cells of the present invention can achieve the effect of killing tumors and improving the tumor immunosuppressive microenvironment simultaneously in the form of a single drug.

### DETAILED DESCRIPTION OF THE EMBODIMENTS

The present invention will be further described below in conjunction with specific embodiments. However, these specific embodiments should not be construed as limiting the protection scope of the present invention. Those skilled in the art can make various changes or modifications to these specific embodiments without departing from the scope of the technical solution of the present invention, and the modified and changed embodiments still fall within the protection scope of the present invention.

The lentiviral vectors and lentiviral packaging plasmids used in the examples of the present application were purchased from commercial companies or synthesized by commercial companies. The gene fragments used in the examples of the present application, including signal peptides, antibody binding regions, hinge regions, TCR constant regions, tag proteins, etc., were synthesized from commercial companies. The embodiments of the present application are only for further describing the present invention and are not intended to limit the scope of the present invention.

### Optimization of STAR

The secreted antibody (Antibody, Ab) or B-cell receptor (BCR) produced by B cells has great similarity to the T-cell receptor (TCR) in terms of genetic structure, protein structure and spatial conformation. Both the antibody and TCR consist of a variable region and a constant region, in which the variable region plays the role of antigen-recognizing and binding, while the constant region domain plays the role of structural interaction and signal transduction. By replacing the variable regions of TCR α and β chains (or TCR γ and δ chains) with the heavy chain variable region (VH) and light chain variable region (VL) of the antibody, VHH or scFv, an artificially synthetic chimeric molecule called Synthetic T-Cell Receptor and Antibody Receptor (STAR) can be constructed.

In some embodiments, a STAR molecule has two chains, wherein the first chain is obtained by fusing an antigen recognition sequence (such as an antibody heavy chain variable region, VH) with a constant region (C α) of a T cell receptor α chain (TCR α), and the second chain is obtained by fusing an antigen recognition sequence (such as an antibody light chain variable region, VL) with a constant region (C β) of a T cell receptor β chain (TCR β). The antigen recognition domain (such as VH, VL or scFv, etc.) and the constant domain (constant domain of TCR α, β, γ and δ) in the construct can be arranged and combined to form a variety of constructs with different configurations but similar functions.

The first and second chains of STAR molecule, after expressing in T cells, will combine with endogenous CD3εδ, CD3γε and CD3ζζ chains in the endoplasmic reticulum to form a eight-subunit complex, which is present on the surface of cell membrane in the form of complex. An immunoreceptor tyrosine-based activation motif (ITAM) is a signal transduction motif in a TCR molecule, with its conserved sequence of YxxL/V. The endodomains of CD3ε, δ, γ and ε chains comprise one ITAM sequence, and that of CD3ζ chain comprises three ITAM sequences, so a complete STAR complex has a total of ten ITAM sequences. When the antigen recognition sequence of a STAR receptor binds to its specific antigen, the intracellular ITAM sequence will be phosphorylated successively, which then in turn activate the downstream signaling pathway, activating transcription factors such as NF-κβ, NFAT, and AP-1, etc., to initiate the activation of T cells and produce effector functions.

Due to the constant region sequences of human, primate and murine TCR α/β chains (mouse TCRAC/mouse TCRBC) are highly conserved, and have the same key amino acid sequence as well, they can be replaced with each other, increasing the efficiency of correct pairing of STAR molecules, reducing the possibility of mispairing leading to unknown specificities, and increasing the safety. The inventors of the present invention have previously modified the constant region of STAR, such as cysteine substitution and transmembrane-domain hydrophobic amino acid substitution, to improve its performance.

1) Modifications of Constant Regions Derived from Mice

For the constant-region sequences of mouse-derived TCRα/β chains, humanization modification, cysteine substitution, and transmembrane-domain hydrophobic amino acid substitution can be carried out.

Introducing Cysteine Point Mutations to Form Disulfide Bonds: the amino acid threonine (T) at position 48 is mutated to Cysteine (C) which is derived from a wild-type mouse TCRα chain constant region, and the amino acid serine(S) at position 56 is mutated to cysteine (C) which is derived from a wild-type mouse TCRβ chain constant region. The two newly added cysteines will form a disulfide bond between the two chains of STAR, thereby reducing the mispairing of the two chains of STAR with endogenous TCR chains and helping the STAR molecule form a more stable complex. The obtained α-chain constant region is named TRAC(Cys), and the obtained β-chain constant region is named TRBC(Cys).

Design of Hydrophobic Amino Acid Substitution in the STAR Transmembrane Region: three amino acid mutations were carried out at amino acid positions 111 to 119 in the transmembrane domain of TCR α chain constant region: serine(S) at position 112 was mutated to leucine (L), methionine (M) at position 114 was mutated to isoleucine (I), and glycine (G) at position 115 was mutated to serine (V). The whole amino acid sequence in this region was changed from LSVMGLRIL (SEQ ID NO:65) to LLVIVLRIL (SEQ ID NO:66). This design increased the hydrophobicity of transmembrane domain, counteracts the instability caused by positive charges carried by the TCR transmembrane domain, and makes STAR molecule more stable on the cell membrane, thus obtaining better functions. The α-chain constant region obtained by combining the cysteine mutation and the hydrophobic-region mutation is named TRAC(Cys-TM), and the corresponding β-chain constant region is named TRBC(Cys-TM), wherein TRBC(Cys-TM) is the same as TRBC(Cys).

To further optimize the design of the STAR molecule, a specific rearrangement is carried out on the N-terminal of the STAR molecule's constant region based on the cysteine point mutation of the mouse-derived constant region and the hydrophobic amino acid mutation of the α-chain constant region to obtain better results. Rearrangement means partial sequence deletion and simultaneous humanization mutation of some sequences. The significance of the humanized mutation is to minimize non-human sequences in the STAR molecule while ensuring the function of the STAR molecule to circumvent the possibility that STAR-T cells are rejected by receptors in clinical applications to the greatest extent. Therefore, the 18 amino acids at the N-terminal of the TCR α chain constant region are modified, including the 6th amino acid E is replaced by D, the 13th amino acid K is replaced by R, and the amino acids at positions 15-18 are deleted. The obtained α-chain constant region is named TRAC(Nrec-Cys-TM). Further, the 25 amino acids at the N-terminal of the TCR β chain constant region are modified, including the 3rd amino acid R is replaced by K, the 6th amino acid T is replaced by F, the 9th amino acid K is replaced by E, the 11th amino acid S is replaced by A, the 12th amino acid L is replaced by V, and the amino acids at positions 17, 21-25 are deleted. The obtained β-chain constant region is named TRBC(Nrec-Cys-TM).

The modified TCRα chain constant region is derived from the TCRα chain constant region of rodents (preferably mice, more preferably mouse), and compared with the wild-type TCRα chain constant region of rodents (preferably mice, more preferably mouse), wherein the substitution of the 122nd amino acid K replaced by R. The modified TCRβ chain constant region is derived from the TCRβ chain constant region of rodents (preferably mice, more preferably mouse), wherein the lysine at positions 150, 168, or 170 is replaced by arginine.

In addition, co-stimulatory molecule such as the cytoplasmic region of OX40 can be connected to the C-terminal of the α chain constant region and/or the β chain constant region to further enhance the function of STAR. The co-stimulatory molecule can be connected to the C-terminal of the α chain constant region and/or the β chain constant region through a linker, such as a (G₄S)₃ linker. The constant region connected to the co-stimulatory molecule, in addition to the above-mentioned modifications, can also lack the natural intracellular region compared to the wild-type constant region, which further improves the function of STAR. For example, the α chain constant region can lack the amino acids at positions 136-137; and/or the β chain constant region can lack the amino acids at positions 167-172.

2) Modifications of Constant Regions Derived from Humans

For the constant-region sequences of human-derived TCRα/β chains, one or more of minimal murinization modification, cysteine substitution, transmembrane-domain hydrophobic amino acid substitution, or rearrangement can be carried out, as follows:

Cysteine Point Mutation in the Human-Derived Constant Region of STAR (hereinafter referred to as hcSTAR): the amino acid threonine (T) at position 47 is mutated to cysteine (C) which is derived from a wild-type human TCRα chain constant region, and the amino acid serine (S) at position 56 is mutated to cysteine (C) which is derived from a wild-type human TCRβ chain constant region (this mutation is named Cys2). An additional disulfide bond is formed between the α chain constant region and the β chain constant region of the STAR molecule, reducing the mispairing of the two chains of STAR with endogenous TCR chains, helping the STAR molecule form a more stable complex, and thus obtaining better functions.

Minimal Murinization (abbreviated as MM) of the Human-Derived Constant Region of STAR: In the constant region of the TCRα chain, the 90th P is mutated to S, the 91st E is mutated to D, the 92nd S is mutated to V, and the 93rd S is mutated to P. In the constant region of the TCRβ chain, the 17th E is mutated to K, the 21st serine(S) is mutated to A, the 132nd F is mutated to I, the 135th E is mutated to A, and the 138th Q is mutated to H.

Hydrophobic Amino Acid Substitution (abbreviated as TM) in the STAR Transmembrane Region: Two amino acid site mutations are carried out at the amino acid positions 110 to 118 in the transmembrane domain of the TCRα chain constant region. The 115th serine(S) is mutated to leucine (L), and the 118th glycine (G) is mutated to valine (V). This design increases the hydrophobicity of the transmembrane region, counteracts the instability caused by the positive charge carried by the TCR transmembrane region, enables the STAR molecule to exist more stably on the cell membrane, and thus obtains better functions.

In addition, co-stimulatory molecule such as the cytoplasmic region of OX40 can be connected to the C-terminal of the α chain constant region and/or the β chain constant region to further enhance the function of STAR. The co-stimulatory molecule can be connected to the C-terminal of the α chain constant region and/or the β chain constant region through a linker, such as a (G4S)₃ linker. The constant region connected to the co-stimulatory molecule, in addition to the above-mentioned modifications, can also lack the natural intracellular region compared to the wild-type constant region, which further improves the function of STAR.

In the existing technologies, in order to kill tumors while improving the tumor immunosuppressive microenvironment, patients need to receive separate treatments or take different medications. This not only affects the therapeutic effect of tumor treatment, increases the burden on patients, but also raises the risk of adverse drug reactions. The dual-functional STAR-T cells of the present invention fully utilize their natural multi-chain structure. By attaching an antibody portion targeting a receptor (such as CCR8) on immunosuppressive immune cells to one chain and an antibody portion targeting tumor antigens (such as MSLN or Claudin18.2) to the other chain, they achieve dual functions. Due to the structural advantage, the two antibody fragments do not interfere with each other and can function independently. Moreover, the antibody fragment targeting the receptor on immunosuppressive immune cells can eliminate the immunosuppressive effect of Treg cells and improve the tumor immunosuppressive microenvironment, thereby enhancing the tumor-killing ability of the antibody fragment targeting tumor antigens. In animal models of epithelial cell carcinoma, glioblastoma, and liver cancer, the dual-functional STAR-T cells of the present invention exhibit better anti-tumor effects than traditional CAR-T cells and have no obvious toxic and side effects.

The dual-functional STAR-T cells of the present invention can achieve the effect of killing tumors and improving the tumor immunosuppressive microenvironment simultaneously in the form of a single drug.

### Example 1: Screening of Nanobodies Targeting CCR8

1.1 Immunization of Alpacas with Human CCR8 Protein

Healthy alpacas were immunized with the commercially available extracellular region of human CCR8 protein (100 μg) (purchased from ACRO Biosystems). The adjuvants included complete Freund's adjuvant (CFA, Sigma) and incomplete Freund's adjuvant (IFA, Sigma). The extracellular region of the human CCR8 protein, which was expressed and purified as described above, was diluted with PBS and then mixed with the corresponding adjuvant at a ratio of 5:1. The antigen and the adjuvant were thoroughly mixed to form a stable emulsion. The antigen mixture was drawn into a syringe and injected subcutaneously at multiple points on the neck skin of the alpacas four times (immunization injections were carried out on days 1, 14, 28, and 42 respectively), with 100-200 μL injected at each point and 100 μg of CCR8 antigen each time. On day 53, alpaca blood samples were collected from the ear marginal vein, and the serum was extracted for antibody titer detection. The P/N value of the serum diluted 200,000-fold was greater than 2.

On days 54, 57, and 60:30-40 mL of alpaca blood samples were collected from the hind-leg veins of the alpacas for PBMC isolation.

### 1.2 PBMC Isolation, RNA Extraction, Reverse Transcription, and Phage Library Construction

PBMCs were isolated from the alpaca blood samples obtained in the previous step using Ficoll separation solution (Cytiva, product number 17544202) according to conventional procedures. RNA was isolated from the PBMCs using conventional methods, and cDNA was synthesized using the Invitrogen reverse-transcription kit (Thermo Scientific, product number #K1622).

The VHH sequences were obtained through two rounds PCR, and homologous arms of the phagemid vector were added to both ends of the sequence. The VHH fragments were ligated to the phagemid vector, and then the ligation product was concentrated and purified (gel extraction kit, TIANGEN, product number #matched with 19-03). The purified ligation product was electro-transformed into competent Escherichia coli cells. Helper phages were added for infection and further cultivation. The phages were harvested and concentrated, and their titers were determined.

1.3 Antibody Screening from the Phage Library

The phage library obtained in the above steps was subjected to three rounds of antibody screening, with each round including one positive selection and one negative selection. First, the phages were incubated with antigen peptides, and the phages that could not bind were discarded, and the phages that bound to the antigen peptide were retained. Then, the retained phages were incubated with BSA for negative selection, and the phages that did not bind to BSA were retained.

The phages obtained after three rounds of screening and M13KO7 helper phages were used to co-infect TG1 cells, The infected cells were plated on 2YT-AK plates. Single colonies were picked for phage expansion. The phages were harvested for binding detection to determine the available phages/antibodies. The two obtained antibodies were named NCR801 and NCR802 respectively.

### Example 2: Performance Detection of Nanobodies Targeting CCR8

### 2.1 EC50 Affinity of CCR8 Nanobody

The EC50 value of the affinity of the CCR8 nanobody was determined. 293T-hCCR8, 293T-hCCR4, and 293T cells were cultured until the cell density reached 80%, and then the experiment was started. The cells were added to 96-well plates at a density of 1×10⁶ cells/well with a volume of 100 μL for flow cytometry staining. The antibodies were serially diluted at a starting concentration of 250 nM with a 3-fold dilution ratio, and 11 samples were prepared for each antibody. The prepared antibodies were added to the cells in the 96-well plates, and after staining at 4° C. for 30 min, the antibodies were washed off with PBS. Then, a secondary antibody was added for staining: APC anti-human IgG Fc Antibody (Cat: 410711, Lot: B343074), and the secondary antibody was diluted 1:200. After the staining was completed, the APC fluorescence value was detected by flow cytometry, and draw a curve based on the experimental results and calculate the EC50 value.

As shown in FIG. 1, the EC50 value of the affinity between NCR802 and the CCR8 protein was 0.19 nM. In the affinity detection of NCR801, non-specific binding was found. The sequences of these antibodies are shown in Table 1.

TABLE 1
Sequences of CCR8 Nanobodies

| Antibody | Description | Sequence | SEQ ID NO |
|---|---|---|---|
| NCR801 | CDR1 | GFTFHYYH | SEQ ID NO: 1 |
| | CDR2 | IRSNGDDI | SEQ ID NO: 2 |
| | CDR3 | ARIRTWRRPKEYDY | SEQ ID NO: 3 |
| | Variable | DVQLQESGGGLVQP | SEQ ID NO: 4 |
| | region | GGSLRLSCAASGFT | |
| | | FHYYHMSWVRQAPG | |
| | | KGPEWVSSIRSNGD | |
| | | DILYADSVKGRFTI | |
| | | SRDNAKNMLYLEMN | |
| | | SLKAEDTALYSCAR | |
| | | IRTWRRPKEYDYLG | |
| | | QGTQVTVSS | |
| NCR802 | CDR1 | GFTFNYYH | SEQ ID NO: 5 |
| | CDR2 | IRSNADDT | SEQ ID NO: 6 |
| | CDR3 | ARISAWRRPKEYDY | SEQ ID NO: 7 |
| | Variable | DVQLQESGGGLVQP | SEQ ID NO: 8 |
| | region | GGSLRLSCAASGFT | |
| | | FNYYHMGWVRQAPG | |
| | | KGPEWVSSIRSNAD | |
| | | DTMYADSVKGRFTI | |
| | | SRDNAKNTLYLQMN | |
| | | SLKAEDTALYSCAR | |
| | | ISAWRRPKEYDYLG | |
| | | QGTQVTVSS | |

### 2.2 Specificity Detection of CCR8 Nanobodies by MPA

The MPA developed and used by Integral Molecular is an array composed of more than 5,220 human membrane proteins (covering 94% of human membrane proteins). Each human membrane protein in the MPA has a complete structure and can be expressed in living cells in its native conformation. MPA is an in vitro tool that can rapidly and comprehensively screen the specificity of candidate therapeutic drugs. As shown in FIG. 2, in this study, the MPA detection of the NCR802-Fc antibody was successfully completed, and no off-target effects were detected. The results showed that NCR802 specifically binds to CCR8.

### Example 3: CCR8 STAR

### 3.1 Construction of CCR8 STAR Vector

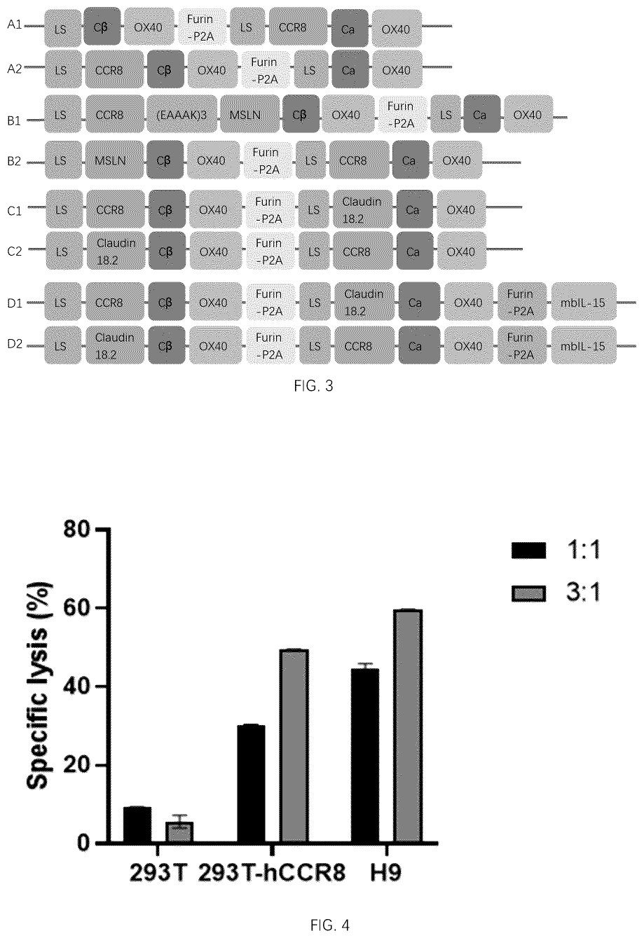

The structure of the STAR targeting CCR8 is shown in FIG. 3 (A1-A2). The sequences of the CCR8 nanobody NCR802 were respectively assembled with the constant regions of the STAR (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is linked directly to the OX40 co-stimulatory domain), and then inserted into a lentiviral vector using the homologous recombination method to construct a complete CCR8-STAR plasmid.

Lentix-293T cells were seeded into 10 cm culture dishes at a density of 5×10⁵ cells/mL and cultured in an incubator at 37° C. with 5% CO₂. When the cell density reached approximately 80% (observed under a microscope), perform transfection. Four plasmids (PMD2.G:PRSV-Rev:PMDlg:transfer plasmid=1:1:2:4) were mixed evenly with 500 μL of serum-free DMEM. 54 μL of PEI-max was mixed evenly with 500 μL of serum-free DMEM and left to stand at room temperature for 5 min (the volume-mass ratio of PEI-Max to the plasmid is 3:1). The PEI-max mixture was slowly added to the plasmid mixture, gently pipetted to mix well, and then left to stand at room temperature for 15 min. The final mixture was slowly added to the culture medium, mixed thoroughly, and then placed back in the incubator for further culture for 12-16 hours. Then, the cells were cultured in 6% FBS DMEM medium, and the virus-containing supernatant was collected at 48 hours and 72 hours.

Jurkat-C4 cells with knocked out TCR were inoculated into flat-bottomed 96-well plates at a density of 1.5×10⁵ cells/mL, and 100 μL of 1640 medium containing 10% FBS and 0.2 μL of 1000×polybrene was added to each well. The virus was serially diluted 10-fold with complete 1640 medium. The diluted cells were added to the virus wells at 100 μL/well, mixed at 32° C., centrifuged at 1500 rpm for 90 min, and then cultured in an incubator at 37° C. with 5% CO₂. After 72 hours, the infection efficiency was measured by flow cytometry. When calculating the titer, wells with an infection rate of 2-30% were selected, and the calculation formula is: Titer (TU/mL)=1.5×10⁴×positive rate÷virus volume (μL)×1000. The above-mentioned virus was used to infect T cells to express STAR.

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus-containing supernatant was added, and the plates were centrifuged at 1500 rpm for 90 min and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

The infection efficiency of STAR was detected, after 72 hours infection, the infection efficiency of STAR was detected by the proportion of the fluorescent tag (RFP).

### 3.2 In Vitro Efficacy Analysis of CCR8 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells.

In the experiment, the NCR802-STAR vector was expressed in T cells, and T cells not infected with the STAR lentivirus (MOCK-T) were used as a reference. Target cells, including 293T, 293T-hCCR8, and H9-LUC, were seeded into 24-well plates at a density of 4×10⁵ cells/well. According to the ratios of CCR8-STAR-positive T cells to target cells of 1:1 and 3:1, the corresponding numbers of STAR-T cells were added to the target cells for co-culture, with a co-culture volume of 1 ml. After 24 hours of co-culture, the co-culture cell suspension was collected.

A luciferase reporter gene detection kit was used to detect the LUC luminescence value, and the killing efficiency of STAR-T cells against the target cells was calculated.

As shown in FIG. 4, NCR802-STAR has good specific killing and specific recognition of target cells. The highest specific killing was observed for the H9 target cells, followed by significant specific killing of the 293T-hCCR8 target cells.

### 3.3 In Vivo Efficacy Analysis of CCR8 STAR

NPG immunodeficient mice were used to establish a model. These mice lack T cells, B cells, and NK cells, and their macrophages and dendritic cells are also defective. In this experiment, 6-8-week-old female NPG mice were used, and the weight difference of mice in each batch was controlled within 2 g. The mice were housed in individually ventilated cages under specific-pathogen-free (SPF) conditions, provided with normal diet and drinking water with a slightly acidic pH to prevent pathogen contamination. All animal operations were carried out after the approval of the Animal protocol for experimental animal research and use.

To verify the in vivo killing effect of STAR-T cells and assess potential safety issues, an H9-LUC target cell tumor model was established to study the in-vivo efficacy and in-vivo expansion of NCR802-STAR and T cells not infected with STAR (MOCK-T). The fluorescently labeled target cells were reinfused into 6-8-week-old female NPG mice via the tail vein at a dose of 1×10⁶ cells/mouse. On the 6th day after infusion, NCR802-STAR (dose groups of 1×10⁶ cells per mouse, 2×10⁶ cells per mouse, 4×10⁶ cells per mouse) and MOCK-T cells were infused via the tail vein. Then, on days-1, 5, 10, 14, 19, and 27, the tumor growth was detected by the luciferin substrate-catalyzed luminescence method, and the tumor fluorescence value, body weight change, and peripheral blood flow cytometry (CD3, CD8, mTCRb) were also detected.

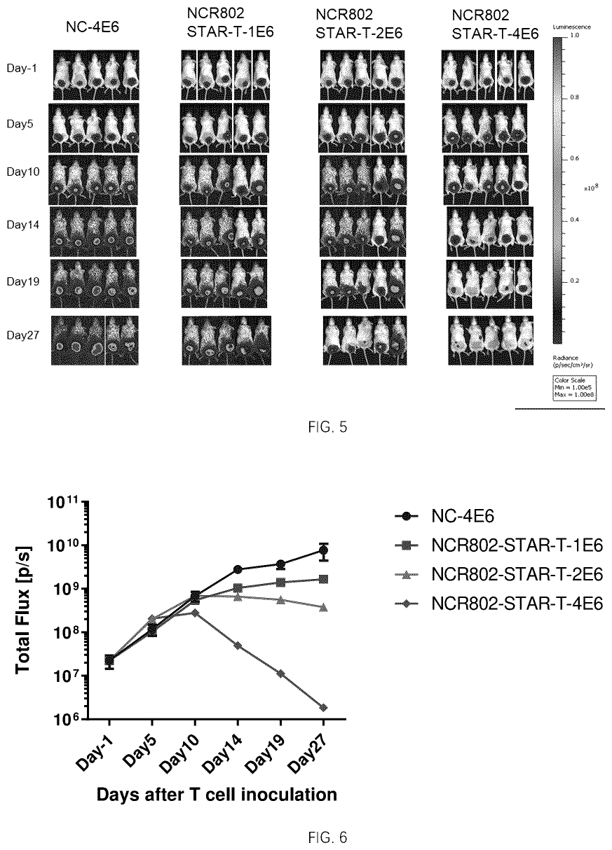

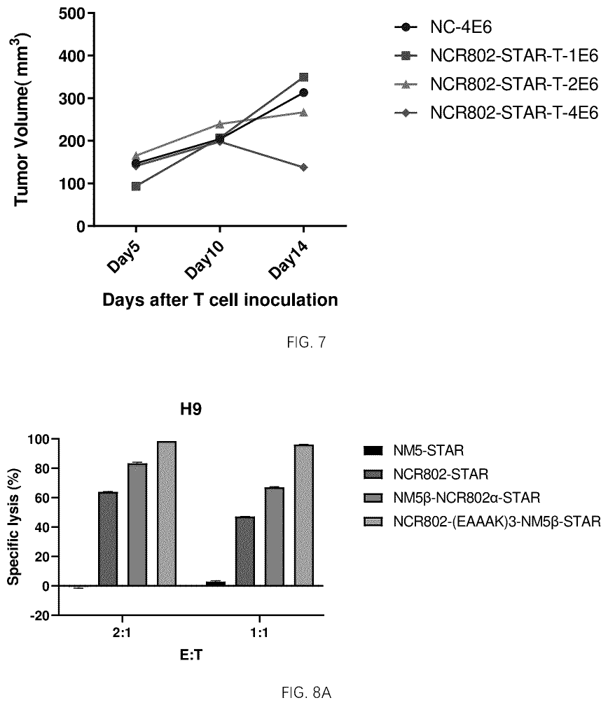

As shown in FIG. 5 to FIG. 7, the NCR802-STAR-T cells in the dose group of 4×10⁶ cells/mouse had a tumor-inhibitory effect on CCR8⁺ target cells. Before 27 days, the NCR802-STAR T cells had a good tumor-inhibitory effect on CCR8⁺ target cells, and the body weight of the mice with these T cells did not decrease significantly, indicating relatively good safety. In terms of survival time detection, the survival time of NCR802-STAR was better, remaining stable in the first 27 days.

### Example 4: Dual Targeting CCR8-MSLN STAR

### 4.1 Targeting MSLN Nanobody and STAR

Previously, the inventors obtained a nanobody NM5 (SEQ ID NO:34) specifically targeting MSLN through a nanobody screening platform. The membrane protein array chip technology was used to demonstrate that the NM5 antibody specifically binds to MSLN, indicating good safety. Through BLI affinity detection and competition experiments, it was found that NM5 has a high affinity for MSLN (KD=1.14×10⁻⁸ M) and has a unique near-membrane end recognition epitope. Based on this, the NM5 STAR was constructed. The α-chain constant region of the NM5 STAR is based on TRAC-Cys-TM, the β-chain constant region is based on TRBC-Cys-TM. The constant regions of both the α-chain and the β-chain are directly connected to the intracellular region of OX40, and the NM5 nanobody is fused to the N-terminal of the β-chain constant region. The amino acid sequence of the α-chain of the NM5 STAR is shown in SEQ ID NO:40, and the amino acid sequence of the β-chain is shown in SEQ ID NO:39. Both the α-chain and the β-chain of the NM5 STAR also contain a GM-CSF signal peptide (SEQ ID NO:38) at the N-terminal, which will be cleaved off in the cells after expression.

### 4.2 Construction of Dual-Target CCR8-MSLN STAR Vector

The coding nucleic acid sequences of the structures shown in FIG. 3 (B1-B2) were commercially synthesized and assembled. The antibody NM5 sequence of MSLN and the antibody NCR802 sequence of CCR8 were respectively assembled with the constant regions of the STAR molecule (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is linked directly to the OX40 co-stimulatory domain), and then inserted into a lentiviral vector using the homologous recombination method to construct a complete CCR8-MSLN STAR plasmid. For the specific method, refer to Example 3.1.

TABLE 2
STAR Targets and Structure Names

| | | STAR Peptide |
|---|---|---|
| | STAR-T | Chains |
| Abbreviation | Structure Name | SEQ ID NO |
| MSLN-STAR | NM5β-STAR- | 39, 40 |
| (NM5 STAR) | abOX40 | |
| CCR8-STAR | NCR802β-STAR- | 49, 50 |
| (NCR802β- | abOX40 | |
| STAR) | | |
| CCR8-MSLN- | NM5β-NCR802α- | 53, 54 |
| STAR | STAR-abOX40 | |
| | NCR802-(EAAAK)3- | 51, 52 |
| | NM5β-STAR-abOX40 | |
| | NM5β-NCR802α- | 53, 54 |
| | STAR-abOX40- | (co-expressed |
| | mbIL-15 | with mbIL-15) |
| | NCR802-(EAAAK)3- | 51, 52 |
| | NM5β-STAR-abOX40- | (co-expressed |
| | mbIL-15 | with mbIL-15) |

### 4.3 Co-Expression Detection of Dual-Target CCR8-MSLN STAR

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus solution was added, and the plates were centrifuged at 1500 rpm for 90 minutes and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

The above-mentioned vectors were used to package lentiviruses and infect T cells. After one week of culture, the expression levels of STAR and MSLN on the cells were detected by flow cytometry. The positive rate of STAR vector infection was detected by mTCRβ. The results showed that NM5 STAR, NCR802 STAR, NM5β-NCR802a STAR, and NCR802-(EAAAK)₃-NM5β STAR (The β-chain is composed in sequence of: the NCR802 antibody, (EAAAK)₃ linker, the NM5 antibody, the TRBC constant region, transmembrane region, and OX40; the α chain is composed in sequence of: the TRAC constant region, transmembrane region, and OX40) could all bind well to the protein.

### 4.4 In Vitro Efficacy Analysis of Dual-Target CCR8-MSLN STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells.

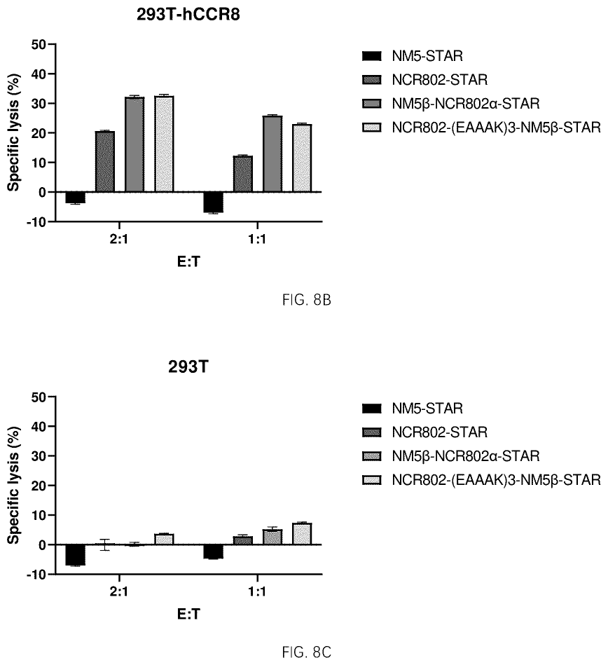

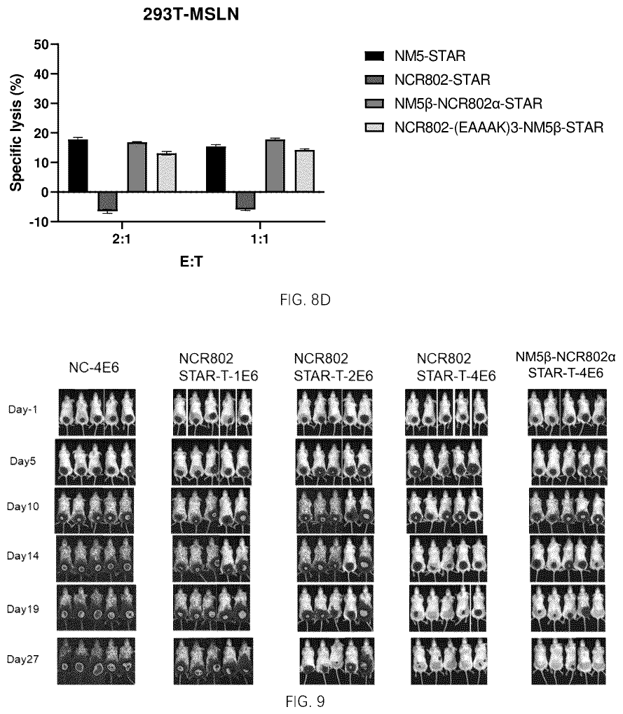

H9, 293T, 293T-hCCR8, and 293T-MSLN target cells were constructed to verify the killing efficiency of CCR8-MSLN STAR-T cells against tumor cells. The above-mentioned target cells were seeded into 24-well plates at a density of 4×10⁵ cells/well. The CCR8-MSLN STAR vector was expressed in T cells. According to the ratios of CCR8-MSLN STAR-positive T cells to target cells of 2:1 and 1:1, the corresponding numbers of CCR8-MSLN STAR-T cells were added to the target cells for co-culture, with a co-culture volume of 1 ml. After 24 hours of co-culture, the co-culture cell suspension was taken, and the LUC luminescence value was detected using a luciferase reporter gene detection kit to calculate the killing efficiency of STAR-T cells against the target cells. As shown in FIGS. 8A-8D, it can be found that:

In the H9 target cell system (FIG. 8A), the T cells of NCR802-STAR, NM5β-NCR802α STAR, and NCR802-(EAAAK)₃-NM5β STAR had a killing effect on the H9 target cells. Among them, the killing efficiency was NCR802-(EAAAK)₃-NM5β STAR-T>NM5β-NCR802α STAR-T>NCR802 STAR-T.

In the 293T-CCR8 target cell system (FIG. 8B), the T cells of NCR802-STAR, NM5β-NCR802α STAR, and NCR802-(EAAAK)₃-NM5β STAR had a killing effect on the 293T-CCR8 target cells. Among them, the killing efficiency was NCR802-(EAAAK)₃-NM5β STAR-T>NM5β-NCR802α STAR-T>NCR802 STAR-T.

In the 293T-MSLN target cell system (FIG. 8D), the T cells of NM5 STAR, NM5β-NCR802α STAR, and NCR802-(EAAAK)₃-NM5β STAR had basically the same killing efficiency on the 293T-MSLN target cells.

In the 293T target cell system (FIG. 8C), none of the STAR-T cells showed in vitro killing.

### 4.5 In Vivo Efficacy Analysis of Dual-Target CCR8-MSLN STAR

To verify the in vivo killing effect of STAR-T cells and assess potential safety issues, an H9-LUC target cell tumor model (CCR8 single-positive target cell model) was established to study the in-vivo efficacy and in-vivo expansion of NCR802-STAR and T cells not infected with STAR (NC). The fluorescently labeled target cells were reinfused into 6-8-week-old female NPG mice via the tail vein at a dose of 1×10⁶ cells/mouse. On the 6th day after infusion, NCR802 STAR-T (dose groups of 1×10⁶ cells/mouse, 2×10⁶ cells/mouse, and 4×10⁶ cells/mouse), NM5β-NCR802α STAR-T (dose group of 4×10⁶ cells/mouse), and T cells not infected with STAR (NC) were infused via the tail vein. Then, on days-1, 5, 10, 14, 19, and 27, the tumor growth was detected by the luciferin substrate-catalyzed luminescence method, and the tumor fluorescence value, body weight change, and peripheral blood flow cytometry (CD3, CD8, mTCRb) were also detected.

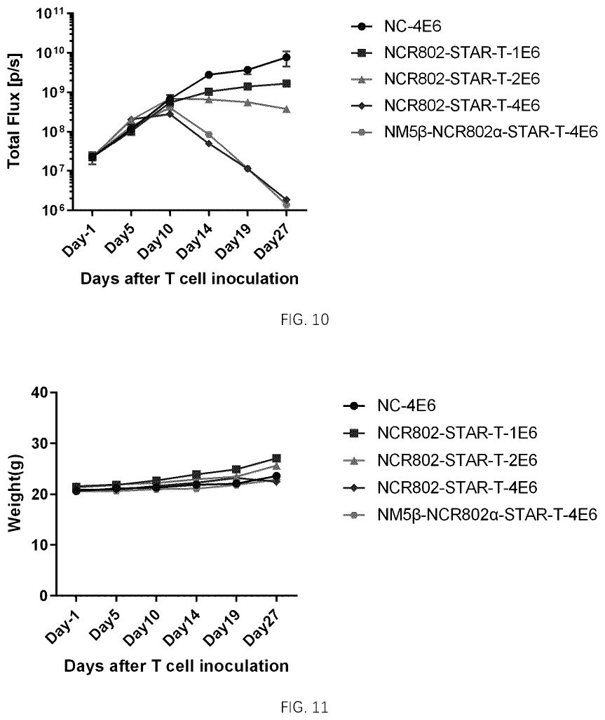

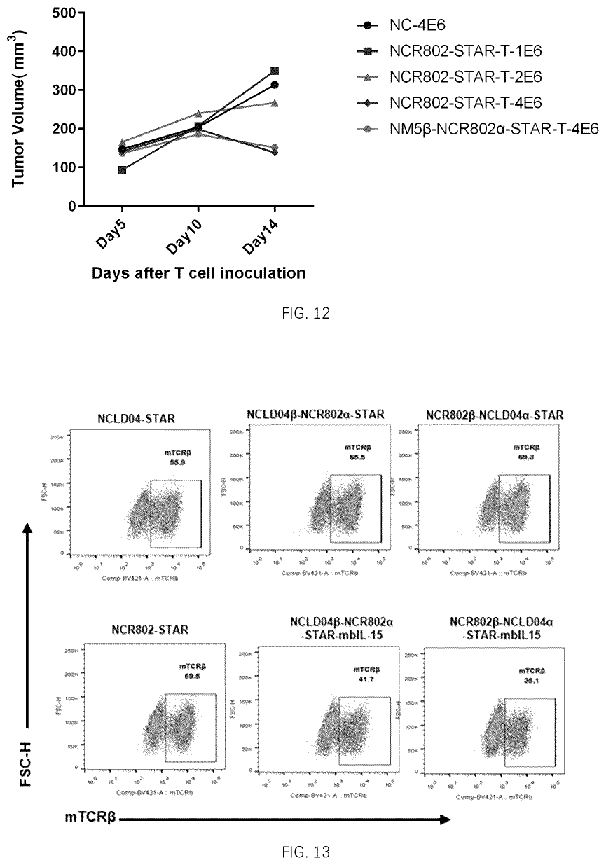

As shown in FIG. 9 to FIG. 12, the NCR802-STAR-T cells in the dose group of 4×10⁶ cells/mouse had a tumor-inhibitory effect on CCR8⁺ target cells. Before 27 days, the NCR802-STAR T cells had a good tumor-inhibitory effect on CCR8⁺ target cells, and the body weight of the mice with these T cells did not decrease significantly, indicating relatively good safety. In terms of survival time detection, the survival time of NCR802-STAR was better, remaining stable in the first 27 days. In the in vivo H9-LUC target cell model, the killing effect of NM5β-NCR802α STAR-T was basically the same as that of NCR802-STAR-T.

### Example 5: Screening of Dual-Target and mbIL-15 Co-Expressed CCR8-Claudin18.2 STAR

### 5.1 Targeting Claudin18.2 Nanobody and STAR

The present invention obtained a nanobody NCLD04 targeting Claudin18.2 and constructed it into NCLD04-STAR-T cells.

A dual-target CCR8-Claudin18.2 STAR-T was constructed in a similar manner to Example 3.

This dual-target CCR8-Claudin18.2 STAR-T had stronger anti-tumor effects both in vitro and in vivo.

### 5.2 Vector Construction and Virus Packaging

Referring to Example 3, the Claudin18.2-CCR8-STAR structure targeting both Claudin18.2 and CCR8 was constructed as shown in FIG. 3 (C1-C2). The sequences of the Claudin18.2 nanobody NCLD04 and the CCR8 antibody NCR802 were respectively assembled with the constant regions of the STAR molecule (The constant regions of the α and β chains of STAR are selected from any one of the sequences of SEQ ID NO: 9-15 and any one of the sequences of SEQ ID NO: 16-22. The α chain is preferably SEQ ID NO: 11 or SEQ ID NO: 14, and the β chain is preferably SEQ ID NO: 18. Moreover, the C-terminus of the constant region of the α and/or β chain of STAR is linked directly to the OX40 co-stimulatory domain), and were inserted into a lentiviral vector using the homologous recombination method to construct a complete Claudin18.2-CCR8-STAR plasmid (Table 3), with MND as the promoter encoding the nucleic acid molecule.

Referring to Example 3, a Claudin18.2-CCR8-STAR structure targeting both Claudin18.2 and CCR8 was constructed. On the basis of this STAR, mbIL-15 was connected via furin-P2A (the vector structure is shown in FIG. 3 (D1-D2)). The nucleic acid sequences encoding the corresponding proteins were synthesized and assembled, and then inserted into a lentiviral vector using the homologous recombination method to construct the STAR-T plasmid. The specific experimental method is described in Example 3.1.

TABLE 3
STAR Targets and Structure Names

| | | STAR Peptide |
|---|---|---|
| | STAR-T | Chains |
| Abbreviation | Structure Name | SEQ ID NO |
| Claudin18.2- | NCLD04β-STAR- | 45, 46 |
| STAR (NCLD04 | a(G4S)3OX40(gh) | |
| STAR) | | |
| CCR8-STAR | NCR802β-STAR- | 49, 50 |
| (NCR802-STAR) | abOX40 | |
| Claudin18.2- | NCLD04β-NCR802α- | 55, 56 |
| CCR8-STAR | STAR-abOX40 | |
| | NCR802β-NCLD04α- | 57, 58 |
| | STAR-abOX40 | |
| | MND-NCLD04β- | 55, 56 |
| | NCR802α-STAR- | (co-expressed |
| | abOX40-mbIL-15 | with mbIL-15) |
| | MND-NCR802β- | 57, 58 |
| | NCLD04α-STAR- | (co-expressed |
| | abOX40-mbIL-15 | with mbIL-15) |

5.3 Co-Expression Detection of Dual-Target and mbIL-15 Co-Expressed CCR8-Claudin18.2 STAR

Primary T cells were obtained by the Ficoll separation method and cultured in X-VIVO medium containing 10% FBS and 100 IU/mL IL-2 at an initial culture density of 1×10⁶/mL. The cells were added to plates pre-coated with CD3, CD28, and Fibronectin for activation. After 24 hours of activation, the virus solution was added, and the plates were centrifuged at 1500 rpm for 90 minutes and then cultured in a CO₂ incubator. After 24 hours of infection, X-VIVO medium containing 10% FBS and 100 IU/mL IL-2 was supplemented, and the cells were transferred to new wells. Subsequently, the cells were passaged every 1-2 days.

The above-mentioned vectors were used to package lentiviruses and infect T cells. After one week of culture, the expression levels of STAR and Claudin18.2 on the cells were detected by flow cytometry. Specifically, the expression of the Claudin18.2 antibody in the STAR structure on the cell membrane was detected by staining with the Claudin18.2 Protein antibody, and the positive rate of STAR vector infection was detected by mTCRβ. The results showed that NCLD04 STAR, NCR802-STAR, NCLD04β-NCR802α STAR, NCR802β-NCLD04a STAR, NCLD04β-NCR802α STAR-mbIL-15, and NCR802β-NCLD04a STAR-mbIL-15 all bound well to the protein (shown in FIG. 13).

5.4 In Vitro Efficacy Analysis of Dual-Target and mbIL-15 Co-Expressed CCR8-Claudin18.2 STAR

Luciferase is a common substance used in cell function research. The activity of luciferase can be determined by adding a luciferase substrate to the system, and the luciferase activity is closely related to the expression of the target gene, the strength of binding, the number of cells, etc. In the present invention, a target cell line stably expressing luciferase was established. The amount of luciferase was used to represent the number of target cells, thereby indicating the killing function of effector cells.

A 293T-Claudin18.2 target cell line, a single-positive target cell line expressing Claudin18.2, was constructed to verify the killing level of CCR8-Claudin18.2 STAR-T cells against tumor cells expressing Claudin18.2. The 293T-Claudin18.2 target cells that only express the Claudin18.2 target were seeded into 24-well plates at a density of 4×10⁵ cells/well. The above-mentioned STAR vectors were expressed in T cells. According to the ratios of positive T cells to target cells of 3:1 and 1:1, the corresponding numbers of STAR-T cells were added to the target cells for co-culture, with a co-culture volume of 1 ml. After 24 hours of co-culture, the co-culture cell suspension was taken, and the LUC luminescence value was detected using a luciferase reporter gene detection kit to calculate the killing efficiency of STAR-T cells against the target cells.

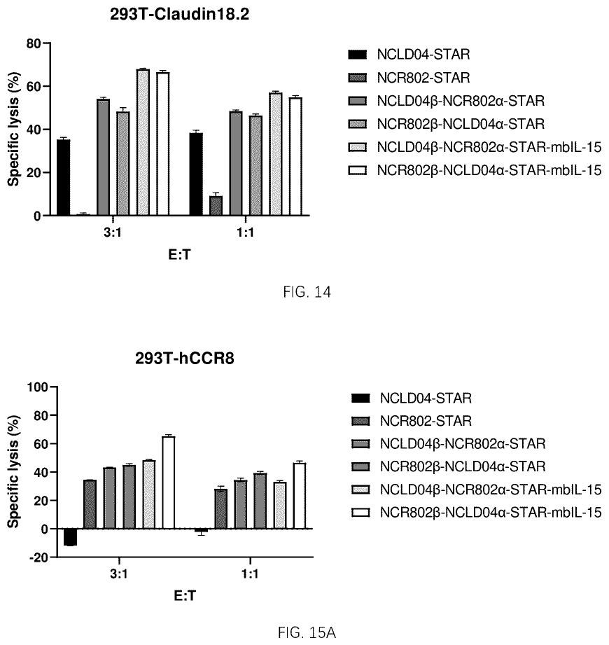

As shown in FIG. 14, in the system of 293T-Claudin18.2 target cells (Claudin18.2 single-positive target cells), NCLD04-STAR, NCLD04β-NCR802α-STAR, NCR802β-NCLD04α-STAR, NCLD04β-NCR802α-STAR-mbIL-15, and NCR802β-NCLD04α-STAR-mbIL-15 all had good killing efficiency against the target cells, and the STAR-T cells co-expressing mbIL-15 had a stronger killing effect than dual-target or single-target STARs.

CCR8 single-positive target cell lines, 293T-hCCR8 and H9 cells, were constructed to verify the killing level of CCR8-Claudin18.2 STAR-T cells against tumor cells expressing CCR8. The 293T-Claudin18.2 target cells and H9 target cells were seeded into 24-well plates at a density of 4×10⁵ cells/well respectively. The above-mentioned STAR vectors were expressed in T cells, and the STAR-T cells were co-cultured with the target cells at ratios of 3:1 and 1:1, with a total co-culture volume was 1 mL. After 24 hours of co-culture, the co-culture cell suspension was taken, and the LUC luminescence value was detected using a luciferase reporter gene detection kit to calculate the killing efficiency of STAR-T cells against the target cells.

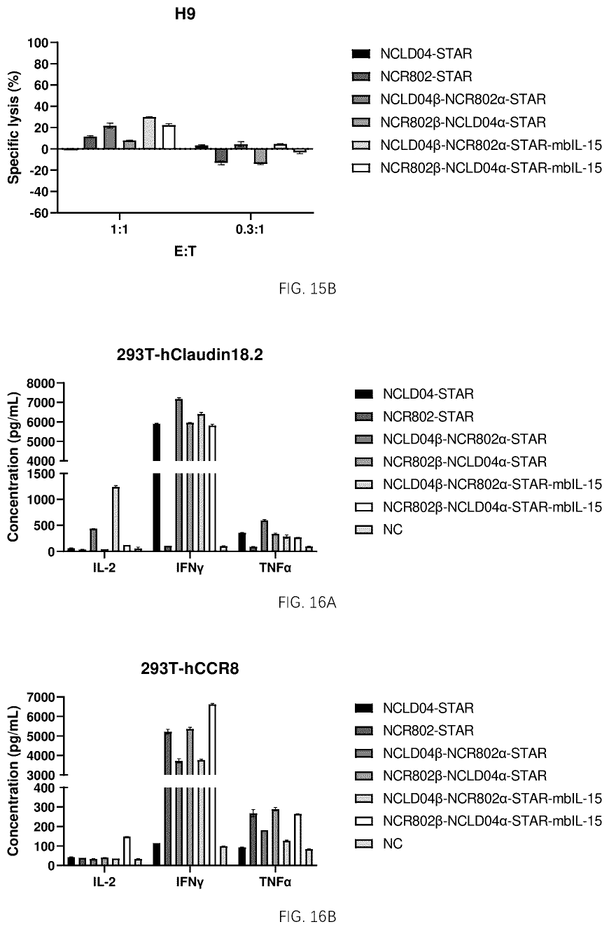

As shown in FIG. 15A, in the 293T-hCCR8 cell line (CCR8 single-positive target cells), NCR802-STAR, NCLD04β-NCR802α-STAR, NCR802β-NCLD04α-STAR, NCLD04β-NCR802α-STAR-mbIL-15, and NCR802β-NCLD04α-STAR-mbIL-15 all had good killing efficiency against the target cells, and the STAR-T cells co-expressing mbIL-15 had a stronger killing effect than dual-target or single-target STARs.

When the effector-to-target ratio of the H9 cell line (CCR8 single-positive target cells) was 1:1 (FIG. 15B), NCR802-STAR, NCLD04β-NCR802α-STAR, NCR802β-NCLD04α-STAR, NCLD04β-NCR802α-STAR-mbIL-15, and NCR802β-NCLD04α-STAR-mbIL-15 all had good killing efficiency against the target cells.

5.5 Cytokine Secretion of Dual-Target and mbIL-15 Co-Expressed CCR8-Claudin18.2 STAR

After the above experiments, the supernatants were collected after co-culturing the above-mentioned T cells and target cells. The secretion levels of IFN-γ, IL-2, and TNF-α were detected by ELISA.

During the activation of T cells, a large number of cytokines are released to assist T cells kill target cells or promote their expansion. Common cytokines include TNF-α, IFN-γ, and IL-2. After T cells are stimulated by target cells or antigens, the T cells are collected, centrifuged, and the supernatant is taken. The ELISA kits used for TNF-α, IFN-γ, and IL-2 are Human IL-2 Uncoated ELISA, Human TNF-α Uncoated ELISA, and Human IFN-γ Uncoated ELISA (with product numbers 88-7025, 88-7346, and 88-7316 respectively). The specific steps are as follows: Dilute the 10× Coating Buffer to 1× with ddH₂O, add the coating antibody (250×), mix well, and then add to a 96-well plate (specially for ELISA) at a volume of 100 μL per well. Seal it with plastic wrap and leave it overnight at 4° C. Wash it 3 times with 1×PBST (also known as Wash Buffer, which is 1×PBS added with 0.05% Tween 20), 260 μL per well each time. Dilute the 5×ELISA/ELISPOT Diluent to 1× with ddH₂O, add to the 96-well plate at a volume of 200 μL per well, and let it stand at room temperature for 1 hour.

Wash it once with PBST. Dilute the standard curve (the ranges are 2-250, 4-500, and 4-500 respectively). Dilute the samples 20-50 times with 1×Diluent. Add the samples and the standard curve, 100 μL per well, with two duplicate wells. Incubate at room temperature for 2 hours, then wash 3 times with PBST. Add the Detection antibody diluted with 1×Diluent, incubate for 1 hour, and then wash 3 times with PBST. Then add HRP diluted with 1×Diluent, incubate for 30 minutes, wash 6 times. Add TMB for color development, ensuring the color development time does not exceed 15 minutes. Add 2N H₂SO₄ to terminate the reaction. And detect the absorbance at 450 nm.

As shown in FIGS. 16A-16B, for 293T-hClaudin18.2 target cells, the factor secretion of NCLD04β-NCR802α-STAR was significantly higher than that of NCR802β-NCLD04α-STAR. Moreover, the combination of NCLD04β-NCR802α-STAR and mbIL-15 could significantly increase the secretion of IL-2. Similarly, for 293T-hCCR8 target cells, the factor secretion of NCR802β-NCLD04α-STAR was significantly higher than that of NCLD04β-NCR802α-STAR, and the combination of NCR802β-NCLD04α-STAR and mbIL-15 could significantly increase the secretion of IL-2.

5.6 In Vivo Efficacy Analysis of Dual-Target and mbIL-15 Co-Expressed CCR8-Claudin18.2 STAR

To further verify the in-vivo inhibitory effect of CCR8-Claudin18.2 STAR-T cells in a mouse transplantation model of Claudin18.2-positive tumor cells, SNU1-Claudin18.2 fluorescently labeled target cells were constructed. These target cells were subcutaneously inoculated into 6-8-week-old female NPG mice at a dose of 2×10⁶ cells/mouse. On the 8th day after inoculation, NCLD04-STAR, NCR802-STAR, NCLD04β-NCR802α-STAR, NCR802β-NCLD04α-STAR, NCLD04β-NCR802α-STAR-mbIL-15, and NCR802β-NCLD04α-STAR-mbIL-15 were infused via the tail vein at a dose of 3×10⁶ cells/mouse. Then, on days-1, 4, 7, 14, 21, 27, 34 and 42, the tumor growth was detected by the luciferin substrate-catalyzed luminescence method, and the tumor fluorescence value, and body weight change were also detected.

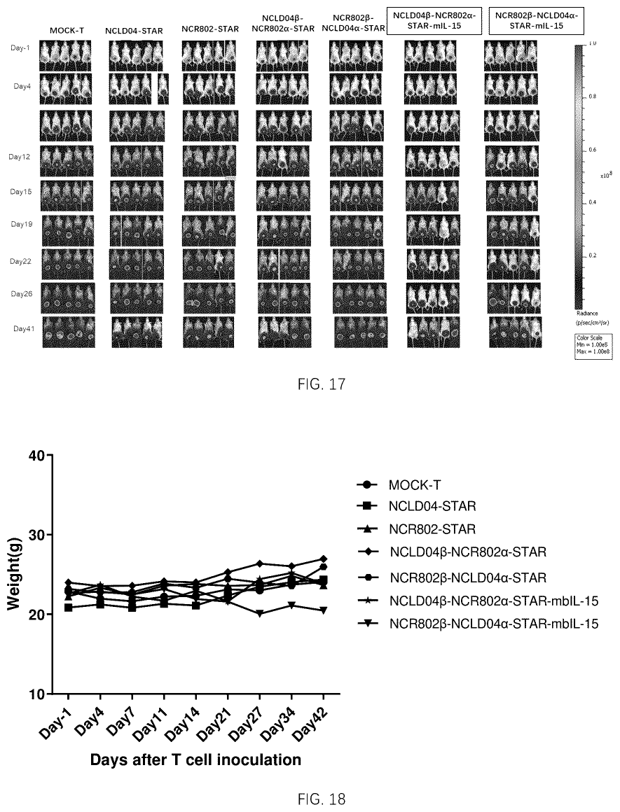

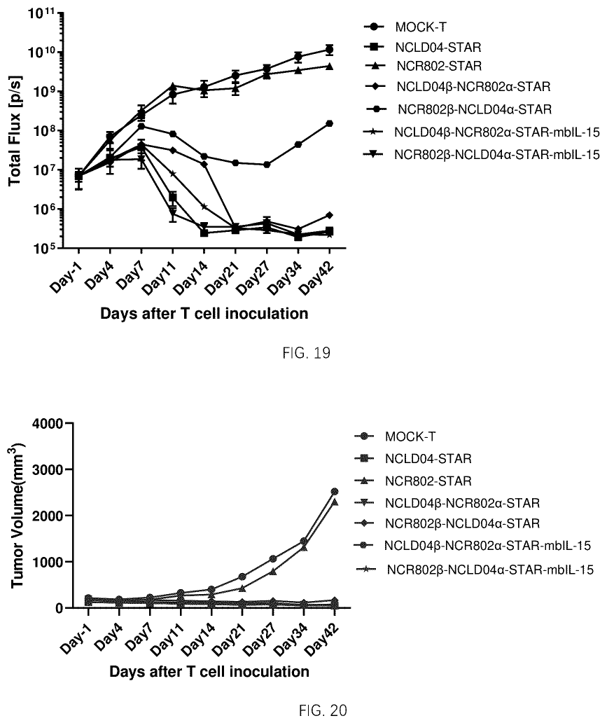

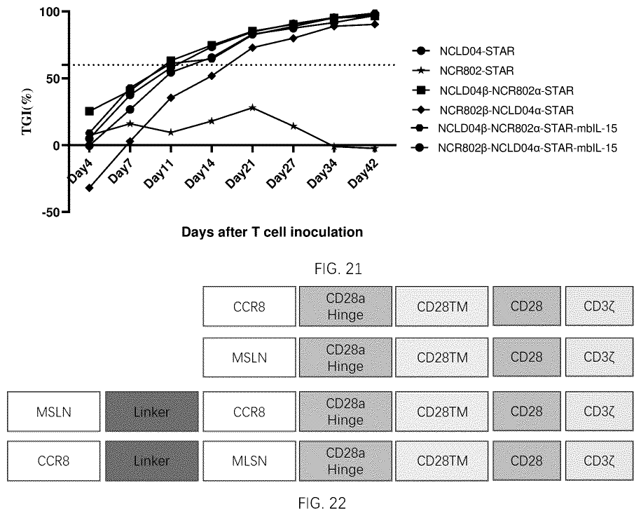

In the test of in-vivo killing effect and potential safety issues in the gastric cancer tumor model, as shown in FIGS. 17, 19-21, NCLD04-STAR, NCLD04β-NCR802α-STAR, NCR802β-NCLD04α-STAR, NCLD04β-NCR802α-STAR-mbIL-15, and NCR802β-NCLD04α-STAR-mbIL-15 all showed good tumor-inhibitory effects on SNU1-Claudin18.2 target cells. Among them, the NCR802β-NCLD04α-STAR-mbIL-15, NCLD04β-STAR, and NCLD04β-NCR802α-STAR-mbIL-15 cells showed the best tumor-inhibitory effects on the target cells, the NCLD04β-NCR802α-STAR and NCR802β-NCLD04α-STAR cells had a moderate tumor-inhibitory effect on the target cells, while NCR802-STAR had the weakest tumor-inhibitory effect on the target cells. Meanwhile, the body weights of the mice treated with these several types of T cells did not show significant decreases (see FIG. 18), indicating relatively good safety. The STAR-T cells with the structures of NCLD04β-NCR802α-mbIL-15 and NCR802β-NCLD04α-STAR-mbIL-15 had the strongest in-vivo expansion ability, could maintain a higher proportion of CD8-positive T cells, but caused higher toxic reactions in mice.

### Example 6: CCR8-CAR-T and Dual-Target CCR8-MSLN CAR-T

### 6.1 Construction of CCR8-CAR-T and Dual-Target CCR8-MSLN CAR Vectors and Virus Packaging

The VHH sequence of the hCCR8-positive antibody NCR802 obtained from the above-mentioned sequencing was assembled with other modules of the CAR molecule (including but not limited to the hinge region, transmembrane region, co-stimulatory domain, signal-transduction domain, VHH region of the antibody targeting the tumor antigen, and/or linker sequences) to construct CARs with different structures, which were then inserted into a lentiviral vector using the homologous recombination method to construct CCR8 CAR plasmids.

The CAR structure targeting both CCR8 and MSLN is shown in FIG. 22. The MSLN nanobody NM5 and the CCR8 antibody NCR802 were respectively connected through linkers such as (G4S)₄, (G4S)₃, or (EAAAK)₃ (SEQ ID NO:25), and then jointly assembled onto the CAR molecular structure. They were inserted into a lentiviral vector using the homologous recombination method to construct a complete CCR8-MSLN-CAR plasmid.

Lentix-293T cells were seeded into 10 cm culture dishes at a density of 5×10⁵ cells/mL. When the cell density reached approximately 80%, transfection was performed. The ratio of the four plasmids was PMD2.G:PRSV-Rev:PMDlg:transformation plasmid=1:1:2:4, and the volume-to-mass ratio of PEI-Max to the plasmid was 3:1. After 12-16 hours, the culture medium was changed, and the virus-containing supernatants were collected at 48 hours and 72 hours respectively.

The virus was serially diluted 10-fold in 96-well plates. Then, Jurkat-C5 cells with knocked-out TCR were added to the virus wells at a density of 1.5×10⁵ cells/mL, 100 μL per well. The plates were incubated at 32° C., centrifuged at 1500 rpm for 90 minutes, and then cultured in an incubator. After 72 hours, the infection efficiency was measured by flow cytometry. Wells with an infection rate of 2-30% were selected for titer calculation, and the titer (TU/mL)=1.5×10⁴×positive rate÷virus volume (μL)×1000.

### 6.2 Detection of Infection Efficiency of CCR8-MSLN CAR-T Cells

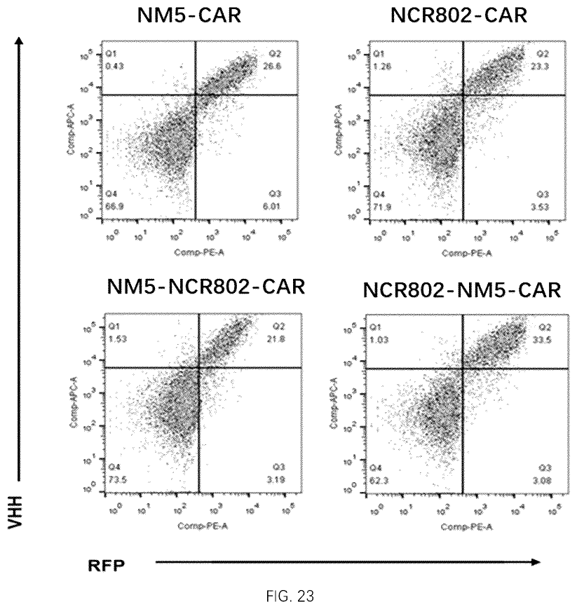

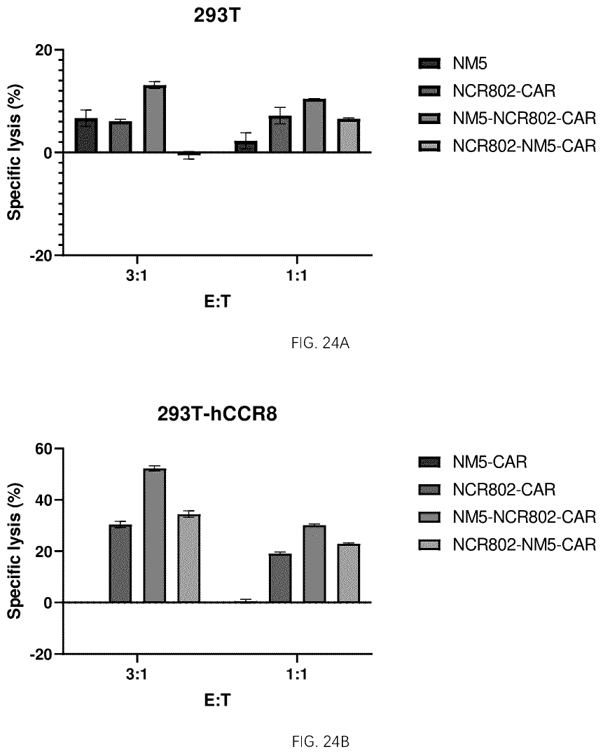

After obtaining PBMCs by the Ficoil separation method, the cells were counted. And then 1.5 times the amount of CD3/CD28 Dynabeads was added and incubated for 45 minutes. The cells were then cultured at a density of 1.2×10⁶ cells/mL. After 24 hours, the cells were infected with the virus at an MOI of 2. Following another 24 hours, the medium was replaced, and the cells were passaged every other day thereafter. At 72 hours post-infection, the infection efficiency of the CAR was analyzed by detecting the proportion of fluorescently labeled (RFP) cells and the binding of VHH antibodies using flow cytometry. The results showed (see FIG. 23) that the single-target NM5-CAR, NCR802-CAR, and the dual-target CCR8-MSLN-CAR could successfully be expressed on the cell membrane, bound well to the protein, and could be used for subsequent experiments.

### 6.3 in Killing Efficiency of CCR8-MSLN CAR-T Cells

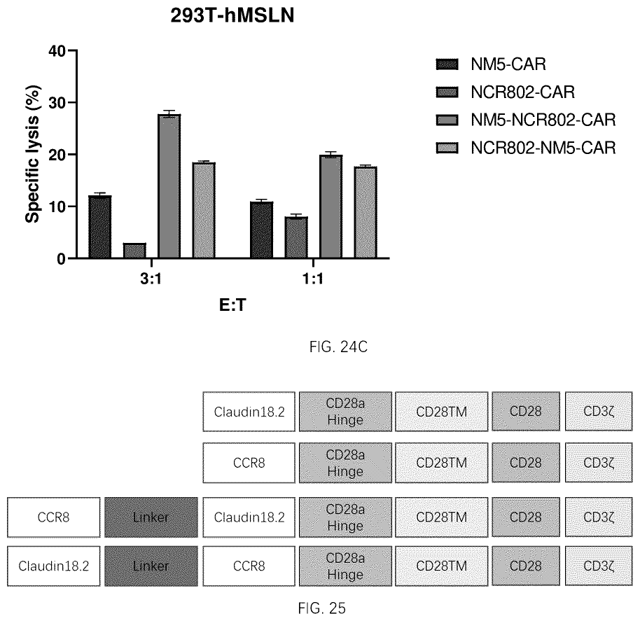

293T cells, 293T target cells overexpressing hCCR8, and 293T target cells overexpressing hMSLN were constructed as target cells. The above three types of target cells were seeded into 24-well plates at a density of 1.5×10⁵ cells/well. After 24 hours, the corresponding numbers of CAR-T cells were added to the target cells at effector-to-target ratios of 3:1 and 1:1 (CAR-positive T cells to target cells), and after 24 hours of culture, the killing efficiency of CAR-T cells against the target cells was detected (see FIGS. 24A-24C).

For the 293T target cells, the killing effects of single- and dual-target CAR-T cells were weak. For the 293T target cells overexpressing hCCR8, both the single-target NCR802-CAR-T and dual-target CAR-T cells had killing efficiency. The killing efficiency of CAR-T cells against the target cells was ranked as follows: NM5-NCR802 CAR and NCR802-NM5 CAR had basically the same killing efficiency, which was greater than that of NCR802-CAR cells.

For the 293T target cells overexpressing hMSLN, all CAR-T cells had killing efficiency. The killing efficiency of CAR-T cells against the target cells was ranked (from high to low) as follows: NM5-NCR802 CAR>NCR802-NM5-CAR>NM5-CAR>NCR802-CAR.

### Example 7: CCR8-CAR-T and Dual-Target CCR8-Claudin18.2 CAR-T

### 7.1 Construction of CCR8-CAR-T and Dual-Target CCR8-Claudin18.2 CAR Vectors and Virus Packaging

The VHH sequence of the hCCR8-positive antibody NCR802 obtained from the above-mentioned sequencing was assembled with other modules of the CAR molecule (including but not limited to the hinge region, transmembrane region, co-stimulatory domain, signal-transduction domain, VHH region of the antibody targeting the tumor antigen, and/or linker sequences) to construct CARs with different structures, which were then inserted into a lentiviral vector using the homologous recombination method to construct CCR8 CAR plasmids.

The CAR structure targeting both CCR8 and Claudin18.2 is shown in FIG. 25. The Claudin18.2 nanobody NCLD04 and the CCR8 antibody NCR802 were respectively connected through linkers such as (G4S)₄, (G4S)₃, or (EAAAK)₃, and then jointly assembled onto the CAR molecular structure. They were inserted into a lentiviral vector using the homologous recombination method to construct a complete Claudin18.2-CCR8-CAR plasmid.

Lentix-293T cells were seeded into 10 cm culture dishes at a density of 5×10⁵ cells/mL. When the cell density reached approximately 80%, transfection was performed. The ratio of the four plasmids was PMD2.G:PRSV-Rev:PMDlg:transformation plasmid=1:1:2:4, and the volume-to-mass ratio of PEI-Max to the plasmid was 3:1. After 12-16 hours, the culture medium was changed, and the virus-containing supernatants were collected at 48 hours and 72 hours respectively.

The virus was serially diluted 10-fold in 96-well plates. Then, Jurkat-C5 cells with knocked-out TCR were added to the virus wells at a density of 1.5×10⁵ cells/mL, 100 μL per well. The plates were incubated at 32° C., centrifuged at 1500 rpm for 90 minutes, and then cultured in an incubator. After 72 hours, the infection efficiency was measured by flow cytometry. Wells with an infection rate of 2-30% were selected for titer calculation, and the titer (TU/mL)=1.5×10⁴×positive rate÷virus volume (μL)×1000.

### 7.2 Detection of Infection Efficiency of CCR8-Claudin18.2 CAR-T Cells

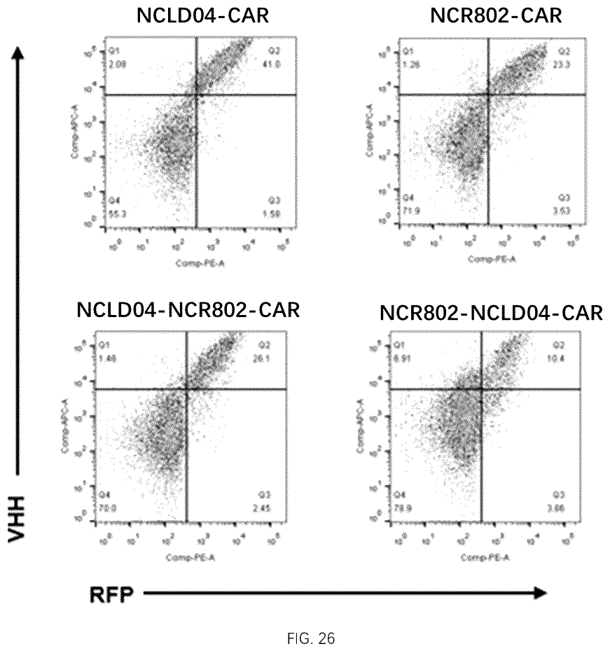

After obtaining PBMCs by the Ficoil separation method, the cells were counted. And then 1.5 times the amount of CD3/CD28 Dynabeads was added and incubated for 45 minutes. The cells were then cultured at a density of 1.2×10⁶ cells/mL. After 24 hours, the cells were infected with the virus at an MOI of 2. Following another 24 hours, the medium was replaced, and the cells were passaged every other day thereafter. At 72 hours post-infection, the infection efficiency of the CAR was analyzed by detecting the proportion of fluorescently labeled (RFP) cells and the binding of VHH antibodies using flow cytometry. All single-target and dual-target CAR-T structures could successfully bind to the cell membrane and had a good binding ability to the protein, which could be used for subsequent experiments (FIG. 26).

### 7.3 in Killing Efficiency of CCR8-Claudin18.2 CAR-T Cells

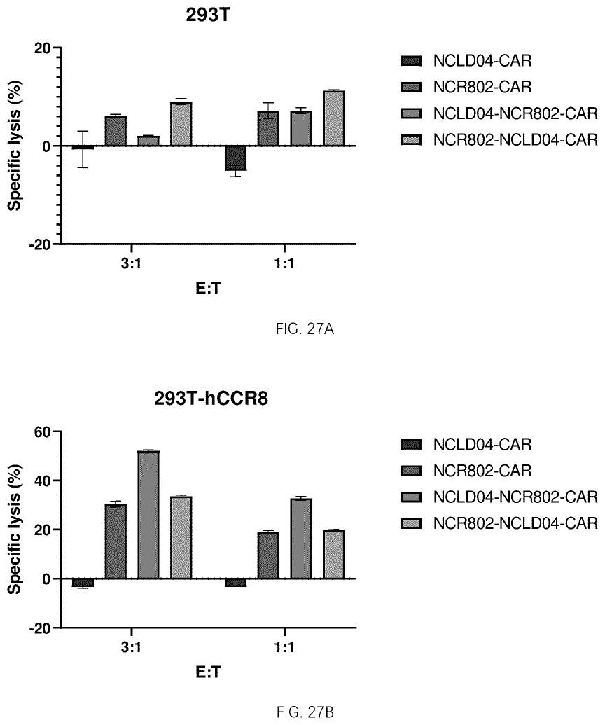

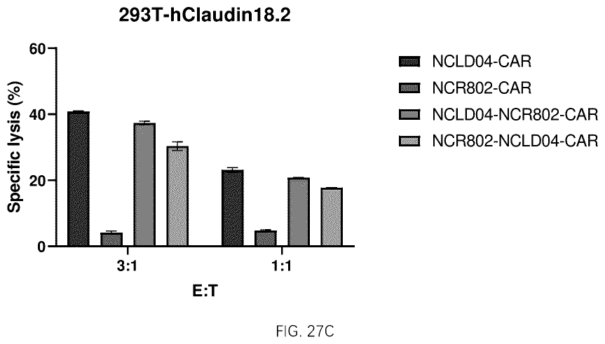

293T cells, 293T target cells overexpressing hCCR8, and 293T target cells overexpressing hClaudin18.2 were constructed. The above three types of target cells were seeded into 24-well plates at a density of 1.5×10⁵ cells/well. After 24 hours, according to the ratios of CAR-positive T cells to target cells of 3:1 and 1:1, the corresponding numbers of CAR-T cells were added to the target cells. After 24 hours of culture, the killing efficiency of CAR-T cells against the target cells was detected (see FIGS. 27A-27C).

For the 293T target cells (FIG. 27A), NCLD04-CAR-T had no killing effect on the target cells, and NCR802-CAR, NCR802-NCLD04-CAR-T, and NCR802-NCLD04-CAR-T had weak killing efficiencies against the target cells.

For the 293T target cells overexpressing hClaudin18.2 (FIG. 27C), both the single-target NCLD04-CAR-T and dual-target CAR-T cells had killing efficiency. The killing efficiency of CAR-T cells against the target cells was ranked as: NCLD04-CAR>NCLD04-NCR802-CAR>NCR802-NCLD04-CAR>NCLD04-CAR, wherein NCR802-CAR-T had no killing effect.

For the 293T target cells overexpressing hCCR8 (FIG. 27B), both the single-target NCR802-CAR-T and dual-target CAR-T cells had killing efficiency. The killing efficiency of CAR-T cells against the target cells was ranked as: NCLD04-NCR802-CAR-T>NCR802-NCLD04-CAR-T and NCR802-CAR-T; NCLD04-CAR-T had no killing effect.

The above-described are only the preferred embodiments of the present invention and do not impose any formal limitations on the present invention. Although the specific implementation manners of the present invention have been described above, those skilled in the art should understand that this is only for illustrative purposes. The protection scope of the present invention is defined by the appended claims. Those skilled in the art can make various changes or modifications to the disclosed technical content without departing from the scope of the technical solution of the present invention, and these changes and modifications all fall within the protection scope of the present invention.

What is claimed:

1. A single-domain antibody that specifically binds to CCR8, comprising CDR1, CDR2 and CDR3 sequences selected from CDR1, CDR2 and CDR3 in SEQ ID NO:8.

2. The single-domain antibody according to claim 1, wherein the CDR1 has an amino acid sequence set forth in SEQ ID NO: 5, wherein the CDR2 has an amino acid sequence set forth in SEQ ID NO: 6, wherein the CDR3 has an amino acid sequence set forth in SEQ ID NO: 7.

3. The single-domain antibody according to claim 1, wherein the amino acid sequence has at least 80%, 90%, 95%, or 99% identity to SEQ ID NO: 8, or wherein the amino acid sequence shown in SEQ ID NO: 8.

4. A synthetic T-cell receptor and antigen receptor (STAR) targeting CCR8, wherein the target-binding region of the STAR comprises the single-domain antibody specifically binding to CCR8 according to claim 1.

5. The STAR according to claim 4, wherein the STAR comprises a first peptide chain and a second peptide chain:
i) the first peptide chain comprises a first target-binding region and a first constant region, and the second peptide chain comprises a second target-binding region and a second constant region; or,
ii) the first peptide chain comprises a first constant region, the second peptide chain comprises a second constant region, and the first peptide chain or the second peptide chain comprises a first target-binding region;
wherein the first target-binding region and/or the second target-binding region comprises one or more antigen-binding regions, and the multiple antigen-binding regions are the same or different;
the antigen-binding region in the first target-binding region and/or the second target-binding region comprises the CCR8 single-domain antibody according to claim 1.

6. The STAR according to claim 4, wherein the STAR comprises any one of the following groups:
a) the first peptide chain comprises a first constant region; the second peptide chain sequentially comprises, from the N-terminus to the C-terminus, at least one CCR8 single-domain antibody according to claim 1, and a second constant region;
b) the first peptide chain comprises, from the N-terminus to the C-terminus, the CCR8 single-domain antibody according to claim 1, and a first constant region; the second peptide chain sequentially comprises, from the N-terminus to the C-terminus, an antibody or antigen-binding fragment specifically binding to MSLN or Claudin18.2, and a second constant region;
c) the first peptide chain comprises a first constant region; the second peptide chain comprises, from the N-terminus to the C-terminus, at least one CCR8 single-domain antibody according to claim 1, an antibody or an antigen-binding fragment specifically binding to MSLN or Claudin18.2, and a second constant region;
In the each of the above group a)-c), the first constant region of the first peptide chain is a TCRα chain constant region or a TCRβ chain constant region, and the second constant region of the second peptide chain is a TCRβ chain constant region or a TCRα chain constant region; the constant regions of the first peptide chain and the second peptide chain are not simultaneously TCRα chain constant regions, nor are they simultaneously TCRβ chain constant regions.

7. The STAR according to claim 6, wherein the first constant region is a TCRα chain constant region or a TCRβ chain constant region or a modified TCRα chain constant region or TCRβ chain constant region;
wherein the second constant region is a TCRα chain constant region or a TCRβ chain constant region or a modified TCRα chain constant region or TCRβ chain constant region.

8. The STAR according to claim 7, wherein the modified TCRα chain constant region comprises an amino acid sequence shown in one of SEQ ID NOs: 11-15, and/or the modified TCRβ chain constant region comprises an amino acid sequence shown in one of SEQ ID NOs: 18-22.

9. The STAR according to claim 6, wherein the first peptide chain and/or the second peptide chain has at least one exogenous intracellular functional domain linked to its C-terminus;
wherein the exogenous intracellular functional domain is an endodomain of OX40, the endodomain of OX40 comprises the amino acid sequence of SEQ ID NO:23;
wherein the exogenous intracellular functional domain is linked directly or via a linker to the C-terminus of the constant region of the first peptide chain and/or the second peptide chain, whose endodomain is deleted.

10. The STAR according to claim 6, wherein the STAR is co-expressed with a membrane-bound IL-15 protein (mbIL-15);
wherein:
i) the amino acid sequence of IL-15 is shown in SEQ ID NO: 27;
ii) the amino acid sequence of the extracellular domain of IL-15Rα is shown in SEQ ID NO: 28;
iii) the amino acid sequence of the linker connecting the extracellular domain of IL-15Rα to IL-15 is shown in SEQ ID NO: 24-25, 29; and/or
iv) the amino acid sequence of mbIL-15 is shown in SEQ ID NO: 30.

11. The STAR according to claim 4, wherein:
a) the STAR comprises a first peptide chain shown in SEQ ID NO:48 and a second peptide chain shown in SEQ ID NO:47;
b) the STAR comprises a first peptide chain shown in SEQ ID NO:50 and a second peptide chain shown in SEQ ID NO:49;
c) the STAR comprises a first peptide chain shown in SEQ ID NO:52 and a second peptide chain shown in SEQ ID NO:51;
d) the STAR comprises a first peptide chain shown in SEQ ID NO:54 and a second peptide chain shown in SEQ ID NO:53;
e) the STAR comprises a first peptide chain shown in SEQ ID NO:56 and a second peptide chain shown in SEQ ID NO:55; or
f) the STAR comprises a first peptide chain shown in SEQ ID NO:58 and a second peptide chain shown in SEQ ID NO:57.

12. A chimeric antigen receptor (CAR) targeting CCR8, comprising an extracellular antigen-binding region, wherein the extracellular antigen-binding region comprises the CCR8 single-domain antibody according to claim 1, and the CAR sequentially comprises, from the N-terminus to the C-terminus, an extracellular antigen-binding region, a hinge region, a transmembrane domain, a co-stimulatory domain, and an intracellular signal-transduction domain.

13. The CAR according to claim 12, wherein the extracellular antigen-binding region further comprises an antigen-binding region specifically binding to another antigen;
wherein the other antigen is Claudin18.2, the antigen-binding region specifically binding to Claudin18.2 comprises CDR1 as shown in SEQ ID NO:41, CDR2 as shown in SEQ ID NO: 42, and CDR3 as shown in SEQ ID NO:43;
or, wherein the other antigen is MSLN, the antigen-binding region specifically binding to MSLN comprises CDR1 as shown in SEQ ID NO:31, CDR2 as shown in SEQ ID NO:32, and CDR3 as shown in SEQ ID NO:33.

14. The CAR according to claim 12, wherein the CAR comprises an amino acid sequence shown in any one of SEQ ID NOs: 59-64.

15. An isolated therapeutic immune cell comprising a STAR or a CAR;
wherein a target-binding region of the STAR or an extracellular antigen-binding region of the CAR comprises the single-domain antibody specifically binding to CCR8 according to claim 1.

16. The therapeutic immune cell according to claim 15, wherein the immune cell is a T cell or natural killer (NK) cell.

17. A method for preparing the therapeutic immune cell according to claim 15, comprising:
step 1) providing a starting immune cell;
step 2) introducing an expression vector into the starting immune cell; and
step 3) harvesting the immune cell obtained in step 2);
Wherein the expression vector comprises an nucleic acid molecule encoding the single-domain antibody, a STAR, or a CAR;
Wherein the single-domain antibody comprises CDR1, CDR2 and CDR3 sequences selected from CDR1, CDR2 and CDR3 in SEQ ID NO:8;
Wherein a target-binding region of the STAR comprises the single-domain antibody;
Wherein an extracellular antigen-binding region of the CAR comprises the single-domain antibody.

18. A pharmaceutical composition comprising a single-domain antibody, a STAR, a CAR, and/or, a therapeutic immune cell, and a pharmaceutically acceptable carrier; Wherein the single-domain antibody comprises CDR1, CDR2 and CDR3 sequences selected from CDR1, CDR2 and CDR3 in SEQ ID NO:8;
Wherein a target-binding region of the STAR comprises the single-domain antibody;
Wherein an extracellular antigen-binding region of the CAR comprises the single-domain antibody;
Wherein the therapeutic immune cell comprises the STAR or the CAR.

19. A method for treating a disease in a subject, comprising administering to the subject a therapeutically effective amount of a single-domain antibody, a STAR, a CAR, a therapeutic immune cell, and/or a pharmaceutical composition;
Wherein the single-domain antibody comprises CDR1, CDR2 and CDR3 sequences selected from CDR1, CDR2 and CDR3 in SEQ ID NO:8;
Wherein a target-binding region of the STAR comprises the single-domain antibody;
Wherein an extracellular antigen-binding region of the CAR comprises the single-domain antibody;
Wherein the therapeutic immune cell comprises the STAR or the CAR;
Wherein the pharmaceutical composition comprises the single-domain antibody, the STAR or the CAR; wherein the disease is a CCR8-related disease, wherein the CCR8-related disease is a CCR8-related autoimmune disease, hematologic tumor, or solid tumor.

20. The method according to claim 19 wherein the autoimmune disease is selected from: systemic lupus erythematosus (SLE), polymyositis and dermatomyositis, systemic scleroderma, Sjögren's syndrome, autoimmune hemolytic anemia, or rheumatoid arthritis;
wherein the hematologic tumor or solid tumor is selected from: epithelial cell carcinoma, glioblastoma, lymphoma, non-small cell lung cancer, leukemia, ovarian cancer, nasopharyngeal carcinoma, breast cancer, endometrial cancer, colon cancer, rectal cancer, gastric cancer, metastatic brain cancer, metastatic liver cancer, lung cancer, digestive tract cancer, bladder cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, mesothelioma, B-cell malignancies or sarcoma.


SEQUENCE LISTING (66 sequences)

SEQ ID NO: 1 - protein, 8 aa, synthetic construct
GFTFHYYH

SEQ ID NO: 2 - protein, 8 aa, synthetic construct
IRSNGDDI

SEQ ID NO: 3 - protein, 14 aa, synthetic construct
ARIRTWRRPKEYDY

SEQ ID NO: 4 - protein, 121 aa, synthetic construct
DVQLQESGGGLVQPGGSLRLSCAASGFTFHYYHMSWVRQAPGKGPEWVSSIRSNGDDILYADSVKGRFTISRDNAKNMLYLEMNSLKAEDTALYSCARIRTWRRPKEYDYLGQGTQVTVSS

SEQ ID NO: 5 - protein, 8 aa, synthetic construct
GFTFNYYH

SEQ ID NO: 6 - protein, 8 aa, synthetic construct
IRSNADDT

SEQ ID NO: 7 - protein, 14 aa, synthetic construct
ARISAWRRPKEYDY

SEQ ID NO: 8 - protein, 121 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSS

SEQ ID NO: 9 - protein, 141 aa, synthetic construct
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 137 aa, synthetic construct
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 11 - protein, 137 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 12 - protein, 133 aa, synthetic construct
DIQNPDPAVYQLRDDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 13 - protein, 135 aa, synthetic construct
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLW

SEQ ID NO: 14 - protein, 135 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLW

SEQ ID NO: 15 - protein, 131 aa, synthetic construct
DIQNPDPAVYQLRDDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLW

SEQ ID NO: 16 - protein, 176 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDF

SEQ ID NO: 17 - protein, 172 aa, synthetic construct
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - protein, 172 aa, synthetic construct
DLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 19 - protein, 166 aa, synthetic construct
DLKNVFPPEVAVFEPSAEIATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 20 - protein, 166 aa, synthetic construct
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 21 - protein, 166 aa, synthetic construct
DLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 22 - protein, 160 aa, synthetic construct
DLKNVFPPEVAVFEPSAEIATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAM

SEQ ID NO: 23 - protein, 37 aa, synthetic construct
RRDQRLPPDAHKPPGGGSFRTPIQEEQADAHSTLAKI

SEQ ID NO: 24 - protein, 15 aa, synthetic construct
GGGGSGGGGSGGGGS

SEQ ID NO: 25 - protein, 15 aa, synthetic construct
EAAAKEAAAKEAAAK

SEQ ID NO: 26 - protein, 27 aa, synthetic construct
RRKRSGSGATNFSLLKQAGDVEENPGP

SEQ ID NO: 27 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 237 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 29 - protein, 26 aa, synthetic construct
SGGGSGGGGSGGGGSGGGGSGGGSLQ

SEQ ID NO: 30 - protein, 377 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 31 - protein, 8 aa, synthetic construct
GVSFSPYG

SEQ ID NO: 32 - protein, 7 aa, synthetic construct
IYRSGST

SEQ ID NO: 33 - protein, 7 aa, synthetic construct
QILGTSY

SEQ ID NO: 34 - protein, 113 aa, synthetic construct
DVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSS

SEQ ID NO: 35 - DNA, 400 bp, synthetic construct
  source 1..400: mol_type = other DNA
aatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcccggtcgccacc

SEQ ID NO: 36 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 37 - protein, 18 aa, synthetic construct
MDWTWILFLVAAATRVHS

SEQ ID NO: 38 - protein, 22 aa, synthetic construct
MLLLVTSLLLCELPHPAFLLIP

SEQ ID NO: 39 - protein, 323 aa, synthetic construct
DVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 40 - protein, 174 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 41 - protein, 8 aa, synthetic construct
GLTFNDYA

SEQ ID NO: 42 - protein, 8 aa, synthetic construct
IRGSGGVT

SEQ ID NO: 43 - protein, 12 aa, synthetic construct
NARPLFGWSEEY

SEQ ID NO: 44 - protein, 119 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSS

SEQ ID NO: 45 - protein, 292 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 46 - protein, 187 aa, synthetic construct
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWGGGGSGGGGSGGGGSRRDQRLPPDAHKPPGGGSFRTPIQEEQADAHSTLAKI

SEQ ID NO: 47 - protein, 209 aa, synthetic construct
DLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 48 - protein, 295 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 49 - protein, 331 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 50 - protein, 174 aa, synthetic construct; identical to SEQ ID NO: 40

SEQ ID NO: 51 - protein, 459 aa, synthetic construct
DVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEAAAKEAAAKEAAAKDVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 52 - protein, 187 aa, synthetic construct; identical to SEQ ID NO: 46

SEQ ID NO: 53 - protein, 323 aa, synthetic construct; identical to SEQ ID NO: 39

SEQ ID NO: 54 - protein, 295 aa, synthetic construct; identical to SEQ ID NO: 48

SEQ ID NO: 55 - protein, 329 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRRDQRLPPDAHKPPGGGSFRTPIQEEQADAHSTLAKI

SEQ ID NO: 56 - protein, 295 aa, synthetic construct; identical to SEQ ID NO: 48

SEQ ID NO: 57 - protein, 331 aa, synthetic construct; identical to SEQ ID NO: 49

SEQ ID NO: 58 - protein, 293 aa, synthetic construct
DVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSSRRDQRLPPDAHKPPGGGSFRTPIQEEQADAHSTLAKI

SEQ ID NO: 59 - protein, 371 aa, synthetic construct
MLLLVTSLLLCELPHPAFLLIPLEDVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 60 - protein, 373 aa, synthetic construct
MLLLVTSLLLCELPHPAFLLIPLEDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 61 - protein, 512 aa, synthetic construct
MLLLVTSLLLCELPHPAFLLIPLEDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSGGGGSGGGGSGGGGSGGGGSDVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - protein, 512 aa, synthetic construct
MLLLVTSLLLCELPHPAFLLIPLEDVQLQESGGGLVQPGGSLRLSCTASGLTFNDYAVSWLRQAPGKEREFVATIRGSGGVTNYGASAEGRFIISRENAENTVYLQMNSLKSEDTAVYYCNARPLFGWSEEYWGQGTQVTVSSGGGGSGGGGSGGGGSGGGGSDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 63 - protein, 506 aa, synthetic construct
MLLLVTSLLLCELPHPAFLLIPLEDVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSGGGGSGGGGSGGGGSGGGGSDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 506 aa, synthetic construct
MLLLVTSLLLCELPHPAFLLIPLEDVQLQESGGGLVQPGGSLRLSCAASGFTFNYYHMGWVRQAPGKGPEWVSSIRSNADDTMYADSVKGRFTISRDNAKNTLYLQMNSLKAEDTALYSCARISAWRRPKEYDYLGQGTQVTVSSGGGGSGGGGSGGGGSGGGGSDVQLQESGGGLVQAGGSLRLSCVGAGVSFSPYGIGWWRQAQGNQREWVASIYRSGSTNYEDFAKDRFTISRDNTKNTAYLQMNSLKPEDTAVYYCQILGTSYRGQGTQVTVSSEFTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 65 - protein, 9 aa, synthetic construct
LSVMGLRIL

SEQ ID NO: 66 - protein, 9 aa, synthetic construct
LLVIVLRIL